(19) United States
(12) Patent Application Publication
(10) Pub. No.: US 2020/0216892 A1
(43) Pub. Date: Jul. 9, 2020

(54) FORCE BASED SEQUENCING OF BIOPOLYMERS

(71) Applicant: Lab79 Technologies, Inc., Boulder, CO (US)

(72) Inventors: Jason Paul CLEVELAND, Boulder, CO (US); Allison Beth CHURNSIDE, Boulder, CO (US); Jan Håkan HOH, Boulder, CO (US)

(73) Assignee: Lab79 Technologies, Inc., Boulder, CO (US)

(21) Appl. No.: 16/735,551

(22) Filed: Jan. 6, 2020

Related U.S. Application Data
(60) Provisional application No. 62/788,710, filed on Jan. 4, 2019.

(51) Int. Cl. C12Q 1/6869; G01N 15/12; G01N 33/487

(52) U.S. Cl. CPC C12Q 1/6869; G01N 33/48721; G01N 15/1245

(57) ABSTRACT

Method and apparatus for determining internal structural properties of a polymer molecule by measuring the force required to translocate the molecule through the interface between two fluids. In some cases, the fluid interface may have a well-defined curvature, which may be held constant or otherwise controlled during translocation. In some cases, the translocating force may be modulated at one or more frequencies. In some cases, attachment of the molecule to a manipulator may be detected before or during translocation.

### INTRODUCTION

Quantification of chemical and physical properties of molecules, macromolecules, nanometer scale and colloidal particles is a generally important problem that has a wide range of useful applications. It is common to examine the properties of a sample in two environments, and by comparing the two situations gain important and useful information about the sample. However, examining the sample as it is translocated between two environments to gain information about the sample is less common. There are a variety of parameters of a sample and the translocation can be quantified during a translocation between two environments.

Here we invoke this concept with a focus on characterizing polymers, in particular nucleic acids and other biopolymers. The identity and order of bases in a nucleic acid molecule (e.g. DNA, RNA) determine what is typically referred to as the sequence of the molecule. The sequencing of DNA and other nucleic acids is important to a wide range of fields. Technologies for nucleic acid sequencing first emerged in the 1970's with the chemical method of Maxam-Gilbert and the enzymatic chain termination-based approach developed by Sanger. Both these methods were “bulk” methods to the extent that they require many molecules and the identification of the base at each position in the sample molecule was determined from the average of group of identical (or very similar) molecules. Sanger sequencing was later automated using capillary gel electrophoresis. This first generation of methods were followed by so-called next-generation sequencing methods that included sequencing by synthesis (and a number of related variants). More recently single molecule methods for nucleic acid sequencing have emerged, including nanopore sequencing and the polymerase-based single molecule methods commercialized by Pacific Biosciences (now part of Illumina, Inc) have emerged. The fundamental detection technologies for these two single molecule methods are respectively, electrical and optical. These methods offer advantages over earlier methods in terms of speed, read length, portability and reagent costs. Although efficient and accurate, sequencing using these methods typically requires a protein or enzyme component that reduces the overall utility of the method because the protein or enzyme loses function or activity after prolonged use or storage. Hence it would be useful to have a single molecule nucleic acid sequencing method that does not require a protein or enzyme.

### BRIEF DESCRIPTION OF DRAWINGS

FIG. 1 is a schematic showing the basic idea of force-based sequencing (FBS). A strand of DNA passes from one fluid to another through an interface. One fluid is shown to contain added molecules and ions, and the interface is decorated with surface active molecules. As the DNA passes through the interface, the different nucleobases adenine, cytosine, guanine, and thymine interact with the interface and with the surface active molecules, resulting in distinct forces. The molecules dissolved in the fluid alter the properties of this interaction.

FIG. 2 is a schematic showing a strand of DNA crossing an interface while force is applied from both ends. One end is held by a magnetic bead, and a force may be applied to it by an external magnetic field.

FIG. 3 shows force based sequencing using lock-in amplification. DNA is pulled through the interface by a tip attached to a cantilever that is being driven at a particular frequency. The cantilever bending is measured using a light source and position detector, using the “optical lever” technique. Force on the cantilever is measured through lock-in amplification of the signal at the drive frequency.

FIG. 4A shows an example for what data from the measurement can look like. After the probe is pulled through the interface, the DNA comes through the interface in a plateau until it detaches.

FIG. 4B is a magnified view of a plateau portion of the graph of FIG. 4A, illustrating that upon closer inspection, the plateau comprises four levels corresponding to the four bases.

FIG. 5 shows a sequencing device based on an array of cantilevers positioned above an array of droplets created by a droplet manipulation layer.

FIG. 6 shows a sequencing device based on an array of cantilevers positioned above an array of droplets created by a microfluidic system.

FIG. 7 shows a device using magnetic beads to control translocation of DNA from droplets. The speed of the magnetic beads, and thus the motion of the DNA through the interface, is controlled by magnets mounted on a motion controller. Light scattered from the magnetic beads lands on a camera, and the image is processed in order to determine their motion.

FIG. 8 shows measurement of the curvature of the interface during translocation.

FIG. 9 depicts steps in an exemplary method of sequencing a polymer, according to aspects of the present teachings.

FIG. 10 depicts steps in another exemplary method of sequencing a polymer, according to aspects of the present teachings.

FIG. 11 depicts steps in yet another exemplary method of sequencing a polymer, according to aspects of the present teachings.

### DETAILED DESCRIPTION

### 1. Overview

In its most general form, the current invention is composed of an interface formed by two fluids (either liquid or gas) in contact with each other. A polymer sample is attached to a translation system that applies a force to the sample, causing it to be translocated across the interface between the fluids. The force required to translocate the sample is measured by the translation system itself, or independent means. Chemical or physical heterogeneity of the sample along the axis of translocation causes variations in the force required for translocation. This setup may then be repeated many times in parallel to increase the overall throughput. The force recordings produced during translocation are analyzed to reveal details of the structure and chemistry of the sample. A particular application is the determination of the sequence of monomers in a biopolymer such as deoxyribonucleic acid (DNA) or ribonucleic acid (RNA). While DNA is used as an exemplary model of a biopolymer in the rest of the patent, it is understood that the invention can be applied to any nucleic acid, including RNA and non-natural ones such as xenonucleic acids (XNA), peptide nucleic acids (PNA), or locked nucleic acids (LNA). Nucleic acids may be composed of natural bases, synthetic bases, unnatural bases, modified bases or some combination thereof.

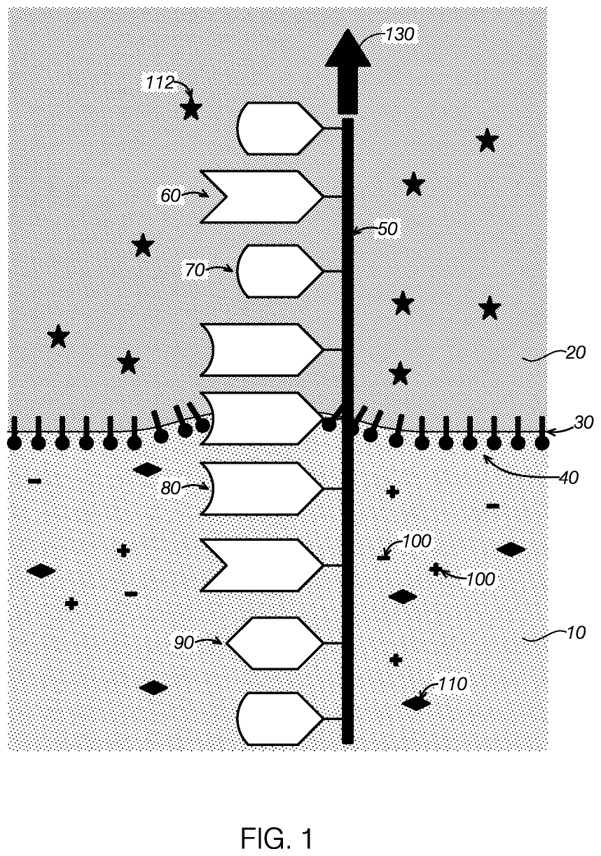

FIG. 1 shows in detail the interface configuration for one embodiment of the invention. Fluid 1 10 and Fluid 2 20 are in contact so that they form an interface 30, which may be decorated with surface active molecules 40; for instance, a surfactant. Ions 100 and molecules 110 can be added to Fluid 1 10, which interact with the DNA 50 and the interface 30. Added molecules may be organic or inorganic, biologically occurring or artificially synthesized. DNA 50 is then pulled through the interface 30 with an applied force 130 and changes in the force as the DNA 50 translocates the interface are monitored. Additional molecules 112 may also be added to Fluid 2 20. The four nucleobases adenine 60, cytosine 70, guanine 80, and thymine 90 interact differently with the interface 30, surface active molecules 40, ions 100, and molecules 110 and 112, producing discernable forces. Typically, Fluid 1 10 is an aqueous phase and Fluid 2 20 is either an air or oil phase.

### 1.1 Interfacial Configuration

A variety of configurations made be used to allow the fluids to contact one another, creating the interface, and to provide access for the force manipulator and detection systems.

### 1.1.1 Interfaces at Openings

Interfaces may be made at openings in solid materials. In one embodiment, a needle or micropipette is introduced into Fluid 2 and Fluid 1 is pushed to the end of the needle or micropipette, forming an interface at its tip. In another embodiment, Fluid 1 and Fluid 2 are separated by a barrier composed of a membrane, plate, solid, gel or slab, and the interfaces are in openings in the membrane. This barrier may be made from graphene, silicon, silicon nitride, gallium arsenide, a ceramic, a metal, a polymer, or some other material. Its surface properties may be altered, for instance by vapor deposition of another material. The material may be treated or passivated as to reduce or alter its reactivity, binding or association with nucleic acids or other soluble or insoluble components of the fluids (including the fluids themselves). These openings in the barrier may be fabricated using lithographic techniques known in the art, for instance using mask and etch steps; by direct ablation or removal of the material using a laser, electron beam, or ion beam; by mechanical drilling; molding; or by other means.

In some embodiments, openings in hydrogel; paper; filters or membranes such as those made of PTFE; or any other material that is porous due to its mesh-like structure, are used as openings containing interfaces. These openings may be <20 nm, <200 nm, <2 um, <20 um, <200 um, <2 mm in size.

For any opening configuration, opening dimensions may be selected so that the resonant frequencies of the interface are above the frequency of the measurement, or for other desirable properties. The resonant frequencies of the interface are important in two respects. The first is that the resonant frequencies determine immunity from ambient vibrational noise. In general, the smaller the size of the interface, the higher the resonant frequencies and the higher the immunity to ambient vibrations. The second is that the interface undergoes thermal fluctuations. While the magnitude of the thermal fluctuations is set by the surface tension of the interface, the resonant frequencies determine at what frequencies the fluctuations occur. By choosing an opening size that pushes the resonant frequencies well above the measurement bandwidth, those fluctuations appear as high frequency noise in the measurement and an average value of the force can be determined.

Further, there is theoretical and experimental evidence that the stiffness of a confined interface depends both on the surface tension between the two liquids and also the size of the confined area relative to the size of the object being pulled through the interface (Dupre de Baubigny et al). Thus the stiffness of the system and size of the forces generated as a molecule is translocated across the interface can also be affected by controlling the dimensions of the opening.

In cases where the opening is connected to a bulk reservoir of much larger size than the opening it can be advantageous to couple the opening to the reservoir through a long, narrow connection. The high friction in such a configuration can help to shield the interface from the low frequency fluctuations present in the reservoir.

In one embodiment, the pressure on the interface is maintained so that the droplet neither grows nor shrinks, or so that it changes size in a controlled manner. The pressure on the interface in an opening-based configuration can also control its radius of curvature.

Openings where interfaces are formed may be a variety of shapes. They can be round, oval, square, rectangular, polygonal or some combination thereof. Openings may also be symmetric, asymmetric or irregularly shaped. Openings may be bounded or unbounded on one or more portions. For example, two plates brought into close proximity would form an opening with a small dimension defined by the distance between the plates and a large dimension defined by the length of the plates or the distance between spacers used to separate the plates. Openings maybe be planar with respect to the surface of the material in which they are formed, or they may be non-planar as to improve the shape of the interface formed for the intended application. For example, the edges of the opening maybe be tapered in one direction of the other, or different parts of the opening maybe be tapered in different directions. Round openings form interfaces that have a single radius of curvature, while other shapes may have multiple radii of curvature. The openings can have a smallest dimension of <2 nm, <20 nm, <200 nm, <2 μm, <20 μm, <200 μm, <2 mm, in combination with a largest dimension of <2 nm, <20 nm, <200 nm, <2 μm, <20 μm, <200 μm, <2 mm.

### 1.1.2 Interfaces by Density Separation

In one embodiment, one fluid is denser than the other fluid. Then the less dense fluid may float on top of the denser fluid under the force of gravity, or, alternatively, the chamber containing the fluids is spun, for instance on a disc, and the denser fluid is pulled to the outside of the disc.

### 1.1.3 Microfluidic Laminar Flow

Microfluidic devices are commonly fabricated in which two or more liquids flow parallel to each other with very little mixing, due to the laminar nature of the flow. This is achieved by joining two or more inputs into one channel, typically in a “Y” configuration. In one embodiment, the interface is formed by two or more liquids introduced by combining two or more streams in a microfluidic device. Common materials for microfluidic devices include semiconductors, such as silicon; polymers, such as polymethyl methacrylate (PMMA); and soft materials such as hydrogels. Many methods for fabricating microfluidic devices using materials such as these are known in the art. The flow through the microfluidic device may either stop before the measurement begins, or it may continue during the measurement. In one embodiment, the molecule under study is pulled across the interface perpendicular to the direction of flow. In one embodiment, the molecule is attached to one side of the chamber and to a force transducer on the other side before flow begins. One or both edges of the microfluidic chamber may serve as a manipulator or detection system, for instance by microfabrication of cantilevers (supported on one end), beams (supported on both ends), or some other structure, into the wall of the microfluidic device. The interface can be moved across the molecule by increasing and decreasing the flow rate on the different inputs. In one embodiment, the two or more liquids are miscible, but do not combine because of the laminar flow.

### 1.1.4 Curved Interfaces

An interface between two fluids may be curved. The radius of curvature may be positive or negative and the magnitude may be <1 nm, <10 nm, <100 nm, <1 um, <10 um, <100 um, <1 mm, <10 mm, <100 mm, <1 cm, <10 cm, <100 cm, <1 m. The interface may be radially symmetric such that there is one value for the principle radii of curvature, or the interface may be radially asymmetric or variable such that the two principle radii of curvature are different. The radius of curvature may be constant before the measurement, or it may vary over the surface of the interface. There are many methods to generate such an interface. As discussed above, if one fluid is contacting another through an opening, the pressure on the fluid may be adjusted to create the desired radius of curvature. This pressure may be applied externally, or it may be a function of the interfacial tension, which is, in turn, determined by the chemical properties of the support structure, each fluid, and any molecules that are present at the surface. In the case of an aqueous fluid, the chemical properties hydrophobicity and hydrophilicity of the support, barrier or walls of the opening may contribute to the curvature of the interface.

Using external pressure applied to a fluid at an opening or other means to change the radius or radii of curvature of a fluid interface also changes the surface tension. The relationship between surface tension and pressure may be describe by the Young-Laplace equation. As noted above, the surface tension is a determinant of the properties of the thermal or mechanically induced fluctuations of the interface, including the magnitude and resonant frequency or frequencies of the fluctuations. Thus these properties can be controlled, manipulated or engineered to improve a force-based measurement by using different combinations of opening shape and dimensions in combination with different applied pressures or radii of curvature. By choosing a combination opening radius and applied pressure that pushes the resonant frequencies well above the measurement bandwidth, those fluctuations appear as high frequency noise in the measurement and an average value of the force can be determined. One of the principle radius of curvature of the interface may be <1 nm, 10 nm, 100 nm, 1 μm, 10 μm, 100 μm, 1 mm, 10 mm, 100 mm, 1 m. A second principle radius of curvature may be <1 nm, 10 nm, 100 nm, 1 μm, 10 μm, 100 μm, 1 mm, 10 mm, 100 mm, 1 m. The curved interface may be formed at an opening with dimensions as described above in 6.2.1, in combination with applied pressures (positive or negative) of at least 0.1 Pa, 1 Pa, 10 Pa, 100 Pa, 1 kPa, 10 kPa, 100 kPa, 1 MPa, 10 MPa, 100 MPa, 1 GPa.

This pressure may also be engineered using a combination of these and other techniques. Besides the advantages mentioned above relative to resonant frequencies, the physical energetics of the interface and especially surface active molecules present at the interface may be tuned by changing the curvatures. As the curvature of the interface increases, the local packing of both the solute molecules at the interface and any surface active molecules changes. At large curvature, the shape of the molecules becomes important as it determines how the molecules pack at the interface. These properties can be optimized to maximize the force differences present when molecules translocate the interface.

An emulsion comprises droplets of a fluid suspended in an immiscible liquid, often stabilized by amphiphilic molecules such as surfactants, in which case the droplets are also known as vesicles. The interface for the FBS device can be made from an emulsion. The emulsion could be made of polar liquid in a nonpolar matrix (e.g. water in oil), or vice versa (e.g. oil in water). Droplets can be created by injection of one liquid into the other, by microfluidic means, by agitation of the solution, or by other means.

In one embodiment, macromolecules to be studied are attached to magnetic beads in solution. This solution is then agitated in an immiscible fluid to form an emulsion, or an emulsion is formed by other means. The droplets will then contain some of the bead-attached molecules. The concentration of the original solution may be selected to achieve the desired number of beads per droplet. In one embodiment, the concentration is selected so that creating a droplet containing two beads is rare. To achieve this, most droplets will have no beads, but those having beads will commonly have only one. In one embodiment, beads containing molecules are selected by magnetic separation. In one embodiment, beads containing molecules are selected by means of optical tweezers. In one embodiment, the emulsion is stabilized with a surfactant, which is chosen to favor the desired droplet size. In one embodiment, the droplets containing magnetic beads are separated from those with no beads by a magnetic field strong enough to move the droplets but too weak to cause the beads to cross the interface. In one embodiment, the droplets themselves are held by optical tweezers while a MEMS tip of the type described elsewhere enters the droplet and pulls the molecule through the droplet. The molecules may be loaded into the droplet similar to the technique described above, either with or without a bead. Alternatively, they may be pre-loaded onto the tip.

Droplets may be attached to a surface. In one embodiment, droplets attached to the surface are held in place while a MEMS type tip, for instance an AFM cantilever, enters the droplet and translocates the molecule through the interface.

Vesicles, or equivalently liposomes, provide another means for engineering a curved interface. A vesicle is a cavity filled with a fluid, enclosed by one or more lipid bilayers. They are distinct from emulsions in that the fluid on the inside and outside are polar (typically aqueous) and only the wall containing the cavity is nonpolar, formed by amphiphilic molecules. For the purposes of this discussion, the wall can be considered as a separate fluid, with an interface on each side. In this view, if the molecule under study is moved from inside the vesicle to outside, it translocates through two interfaces, one on each side of the vesicle wall. The wall itself may be considered to be the interface. Vesicles are common in biology but there are also many techniques to generate them in vitro that are known in the art. Vesicles may be used that are larger than 100 nm, larger than 1 um, larger than 10 um, or larger than 100 um in diameter. Vesicles may be fabricated that use naturally occurring or non-naturally occurring amphiphilic molecules in the wall. The wall may be homogeneous or contain a mixture of different amphiphilic molecules. It may also contain other molecules. For instance, cholesterol may be introduced, which can alter the properties of a lipid bilayer system. Vesicles may be in solution during the measurement, or attached or partially fused to a support surface.

### 1.1.5 Digital Microfluidics

Droplets on a surface may be manipulated using a technique known as “digital microfluidics.” This technique relies on electrowetting, a phenomenon whereby the contact angle of a droplet on a surface decreases when an electric field is applied at the surface, causing the droplet to wet. By adjusting the voltage applied to regions of a surface, droplets can be manipulated: moved, separated, combined, mixed, and so on. In one embodiment of the invention, one fluid is in a droplet that is manipulated by digital microfluidics. This may be before the measurement takes place, as a way of moving the droplets into position for the measurement. Digital microfluidics, and electrowetting in general, may also be used during the measurement, to adjust the curvature of the droplet or to sweep the meniscus across the sample molecule.

Digital microfluidics can be constructed in an open face configuration with an array of electrodes on a single side of the droplet layer, or in a sandwich configuration where there is an additional electrode present and the droplet layer is sandwiched between the two electrode layers. In the sandwich configuration it is often advantageous to make the second electrode from a transparent material to allow optical access to the droplet layer. Such transparent electrodes may be formed from transparent, conductive oxides such as indium tin oxide, ultrathin metal films, graphene, or nanogrids or arrays of nanowires.

### 1.1.6 Multiple Interfaces

Devices are not limited to one interface but can have several sequential interfaces separating chambers of different types of fluid. These chambers can be aligned in a planar configuration, for instance by floating layers of liquids with differing density on top of each other. A set of liquids may, for instance, contain an aqueous solution, then a nonpolar liquid, then another aqueous solution. There can be one, two, three, or more such interfaces. The liquid types can all be different. They can also be repeating layers. For instance, the layers could be a 10 mM NaOH solution, followed by hexadecane, followed by another 10 mM NaOH solution, another layer of hexadecane, and so on. The multiple interfaces can also be round, such as a droplet attached to the surface or floating in solution. As discussed above, a vesicle wall can be considered as a liquid on its own, forming interfaces on both the inner and outer edges. Planar interfaces can also be created with amphiphilic molecules. In one embodiment, lipid bilayer membranes are deposited onto a surface containing wells. It has been shown in the literature that these membranes can be made suspended on top of wells. This again provides two interfaces, one on each side of the lipid bilayer.

In one embodiment, a sample is purified by first pulling it through one interface, then measured by pulling it through another interface. For instance, a device may have three or more chambers with alternating layers of immiscible fluids. The first chamber contains a non-purified sample, for instance in aqueous solution, the second another fluid, and another in the final chamber. The manipulator is translocated through all of the layers into the non-purified sample, where it specifically binds to the desired molecule, leaving all other contaminant molecules behind as it traverses the first interface. It then traverses the second interface, which is where the force is measured. In one embodiment, the first chamber is aqueous, the second a nonpolar liquid such as an oil, the third a denaturing aqueous solution such as a basic solution with pH 11 or higher, and the final chamber contains air. In one embodiment, more than three layers are used in this manner.

### 1.2 Fluids

### 1.2.1 Types of Fluid

The molecule is pulled from one fluid into a second fluid that is immiscible with the first. These could be a wide range of fluids, including gases and liquids. A fluid may refer to a three-dimensional fluid, where molecules are free to move in all three dimensions, such as an aqueous solution. It may also refer to a two-dimensional fluid, where molecules are constrained in one dimension but free to move in the other two, for example a lipid bilayer. Fluids needn't be homogeneous, but can contain a mixture of different fluids and solutes. Generally, if the device comprises two liquid-phase fluids, one fluid will be polar and the other will be non-polar. The device can also comprise a liquid and a gas. In one embodiment, an aqueous solution is used. In one embodiment, that solution contains only monovalent ions. In one embodiment, that solution also contains multivalent ions. In one embodiment, one of the fluids is a hydrocarbon. In one embodiment, that hydrocarbon is an alkane. In one embodiment, the nonpolar fluid is in the solid phase at room temperature, and the device is operated at an elevated temperature, above its melting point. Ionic liquids are salts in their liquid phase. The term often refers to those that are liquid at room temperature. DNAs are soluble in many room-temperature ionic liquids. In one embodiment, one of the fluids is an ionic liquid.

In another embodiment, the second fluid is air, where the humidity is maintained at a low value to limit the hydration of the strand being pulled into the air. Humidity values below 50%, 20%, 10%, 5%, or 1% may be desirable. Alternatively, a dry gas such as helium, nitrogen, or argon is used as the second fluid. In such situations, evaporation of the first fluid may become a problem. However, if the interface is formed by an overpressure pushing the droplet out from an opening as described above, the droplet will be continually replenished as evaporation occurs.

### 1.2.2 Modifying Fluid Properties

One or more of the fluids used can be modified with solutes to facilitate or improve sequencing. The pH of an aqueous fluid has significant influence on the structure and properties of nucleic acids. To control the pH of aqueous fluids, buffers may be used, including the buffers TRIS, HEPES, MOPS, MES, PIPES, Phosphate or acetate. The pH can also be set without a buffer using an acid or a base. The pH of one or more of the fluids may be between 2 and 13. Different fluids used in a single measurement may have different pH.

The polarity of a fluid also plays an important role in determining the properties of a nucleic acid in the fluid. For example, an aqueous fluid can be made less polar by the addition of alcohols such as methanol, ethanol or propanol. Decreasing polarity of an aqueous solution decreases the solubility of DNA, and at low enough polarity DNA will become insoluble. Changes in solubility are reflected in the persistence length and other physical and chemical properties of the DNA. In contrast, non-polar fluids such as tetradecane or hexdecane can be made more polar using alcohols such as octanol or decanol. The range of polarity available can be further extended using mixtures of three different molecules.

### 1.2.3 Sequence-Specific Additions to the Fluid

By adding into the fluid a protein that recognizes sequence from outside of a double stranded structure, sequences can be read on a double strand. The most common of these is the family of zinc finger proteins, which bind into the major groove of a specific DNA sequence. It is common to engineer zinc fingers to bind into a desired sequence. Such a protein can be attached to a specific sequence of DNA in one medium. The second medium is then chosen so that the DNA-Zinc finger interaction will be unstable, leading to it being stripped from the DNA upon translocation. This stripping would produce a higher force, sensed by the force sensor. Sequence specific molecules can stabilize local secondary structure, which is then altered upon translocation, causing a change in the force. This mechanism can be either used to recognize particular sequences, if a full sequencing is not necessary, replacing expensive DNA arrays. DNA arrays are a technology for recognizing specific sequences without fully sequencing the DNA. However, binding molecules can be used to sequence long stretches of DNA as well, by using them either sequentially, or by using molecules that bind different short sequences and also produce different force signatures upon translocation through the interface.

### 1.2.4 Denaturing Fluids

One special case of modifying properties of the fluid and molecules added to it is to denature biological macromolecules with secondary structure into single polymer chains. In one embodiment, a nucleic acid is denatured from its helical shape into single strands prior to translocation across the interface. High pH is one known way to denature DNA. For instance, 10 mM NaOH (sodium hydroxide) is sufficient to destabilize the double helix and produce only single-stranded DNA. In one embodiment, one of the two fluids is a liquid with a pH greater than 8. In one embodiment, one of the two fluids is a liquid with a pH greater than 9. In one embodiment, one of the two fluids is a liquid with a pH greater than 10. In one embodiment, one of the two fluids is a liquid with a pH greater than 11. In one embodiment, one of the two fluids is a liquid with a pH greater than 12. In one embodiment, one of the two fluids is a liquid with a pH greater than 13. In one embodiment, a solvent is used in which a nucleic acid is soluble but the duplex is not stable, producing single-stranded DNA. Examples of such solvents include dimethyl sulphoxide (DMSO), dimethyl formamide, and aqueous solutions of other organic solvents. In one embodiment, heat is used to denature the molecule. The temperature to melt the molecule could be in the range of 30° C. to 100° C. The temperature can be above 100° C. The pressure in the chamber can be one atmosphere, greater than one atmosphere, or less than one atmosphere. In one embodiment, urea is used to denature a macromolecule, for instance a protein. As mentioned below, the presence of ions stabilizes the helix of nucleic acids, so denaturation is more likely in pure water. In one embodiment, one of the fluids is pure water.

### 1.2.5 Ion Association and Binding to Nucleic Acids

Organic and inorganic solutes also associate with nucleic acids in various ways. As indicated in FIG. 1, the association of an ion (100) with a nucleic acid (50) being pulled through an interface (30) will produce a change in the force profile, relative to that of a nucleic acid with a different ion or without an ion. Monovalent ions such as Na⁺ or K⁺ play a role in stabilizing double stranded DNA through dielectric effects as well as direct associations with the molecule. Increasing concentrations of monovalent ions tend to increase the stability of double stranded DNA. Monovalent ions also influence the structure of single stranded nucleic acids as manifest by changes in persistence length. Changes in monovalent ion compositions and concentration of a medium also changes the energetics of partitioning relative to another medium. Thus, the force profile of a nucleic acid translocated through an interface will depend on the type(s) and concentration(s) of monovalent ions in one or more of the fluids. In one embodiment, monovalent ions, including cations or anions, are present in one of the fluids. The ions maybe be selected from Al⁺³, NH₄⁺, Ba⁺², Ca⁺², Cr⁺², Cr⁺³, Cu⁺, Cu⁺², Fe⁺², Fe⁺³, H⁺, H₃O⁺, Pb⁺², Li⁺, Mg⁺², Mn⁺², Mn⁺³, Hg₂⁺², Hg⁺², NO₂⁺, K⁺, Ag⁺, Na⁺, Sr⁺², Sn⁺², Sn⁺⁴, Zn⁺², H⁻, F⁻, CL⁻, BR⁻, I⁻, ASO₄³⁺, ASO₃³⁻, SO₄²⁻, HSO₄⁻, S₂O₃²⁻, SO₃²⁻, ClO₄⁻, ClO₃⁻, ClO₂⁻, OCl⁻, CO₃²⁻, HCO₃⁻, CH₃COO⁻, CN⁻, OCN⁻, SCN⁻, OH⁻, O²⁻, S²⁻, N³⁻, PO₄³⁻, HPO₄²⁻, H₂PO₄⁻, NO₃⁻, NO₂⁻, IO₃⁻, BrO₃⁻, OBr⁻, CrO₄²⁻, Cr₂O₇²⁻, HCOO⁻, NH₂⁻, O₂²⁻, C₂O₄²⁻, MnO₄⁻. The ions may be used at a concentration of 1-100 pM, 0.1-10 nM, 10-1000 nM, 1-100 μM, 0.1-10 mM, 10-1000 mM, 1-10 M.

Divalent ions association is generally stronger than that of monovalent ions, with many divalent ions binding to specific sites on nucleic acids. Divalent metal ion binding for example is base specific, and strength of association with the nucleosides of the 5 “conventional” bases generally decreases according to guanine>cytosine>adenine>(thymine or uracil). Divalent metal ion binding is also ion specific, and for guanine the strength of ion binding decreases according to Pb²⁺>Cb²⁺>Zn²⁺>Cu²⁺>Mn²⁺>Ca²⁺>Mg²⁺. Ion binding occurs in different places on a molecule, such as the phosphates on the backbone, the N7 of guanine or combinations thereof. Thus, ions and combinations of ions can be selected to modify the force profile of a DNA molecule translocating an interface in a sequence dependent fashion. The exact concentrations needed of the ion(s) will depend on the particular composition of the medium selected, but for any given conditions can be determined by experiments in which the concentration(s) are varied until a desirable force profile is achieved. The same methods used to determine ion associations with normal bases can also be used to determine the ion associations with modified bases, including bases with epigenetic or other natural modifications as well as unnatural modifications. Specific combinations of ions in the solution with the nucleic acid may also be used to distinguish these other bases from each other or the 5 conventional bases above.

Trivalent or higher valency ions also associate with or bind to nucleic acids. These include ions such as [Co(NH₃)₆]Cl₃ the polyamine spermine, which can cause nucleic acids to compact into toroidal or rod shaped structures, as well as a variety of more complicated looped structures. These ion induced structures will produce unique force signatures as the nucleic acid is pulled through an interface by the force required to unfold the ion induced structure, the force required to translocate the entire structure, or some combination thereof. Thus, the force-based assay may be used to study or determine structures of nucleic acids induced by trivalent or higher valency ions, as well as any other molecule that binds or associates with a nucleic acid.

### 1.2.6 Other Molecule Additions to Fluid

Other molecules may be added to fluids on one or both sides of the interface. These molecules may change the properties of the fluid; change the energetics of the interface; interact with the DNA, either specifically or nonspecifically; alter the binding of the DNA to the manipulator; change the properties of the detection; allow manipulation; and/or aid any other part of the force based sequencing process. The molecules may be small molecules; biological molecules; artificially synthesized molecules; proteins or peptides; nucleic acids or oligomers; or any other molecule.

Many small molecules associate with nucleic acids through predominantly non-ionic interactions or mixed ionic and non-ionic interactions, and such molecules will also produce a detectable change in the force profile when the nucleic acid is translocated through an interface (compared with the force profile when the small molecules are not there). These include molecules such as the intercalating dyes ethidium bromide, propidium iodide, and YOYO-1. These particular examples act as stains for nucleic acid by virtue of the fact that intercalation between two bases produces a large increase in fluorescence intensity. Many of these molecules have a preference for double stranded DNA, but will also associate with or bind to single stranded nucleic acids. TOTO-1 is a DNA dye that interacts with ssDNA. SYBR gold is another dye that is typically used to stain single stranded DNA in denaturing conditions, for instance gels containing urea and formaldehyde. SYBR green II is another dye that is particularly useful for RNA and can also bind to single-stranded DNA. The association or binding of intercalating dyes to single stranded nucleic acids is typically weaker than with double stranded molecules, although some molecules such as TOTO-1 are reported to have affinities for single stranded nucleic acids similar to those for double stranded nucleic acids. Intercalating dyes have varying degrees of sequence specific, ranging from almost none to dyes such as ACMA (9-amino-6-chloro-2-methoxyacridine) which has a high preference to d(AT) stretches of DNA. Thus, certain intercalators can be used alone or in combinations to identify specific nucleic acid sequence. Certain drugs are known to bind nucleic acids. For instance, quinolones are a class of antibiotics that have been shown to bind single stranded oligonucleotides in a sequence-dependent manner. Organic small molecules can be discovered to bind particular sequences, optimized for the task of binding specific ssDNA sequences, building upon what is known about existing single stranded binding small molecules. Other nucleic acids bind biological macromolecules, including DNA and RNA. These could also be nucleic acids with non-natural backbones (XNAs) or modified bases. Aptamers and Somamers can be discovered using the SELEX process to bind nearly any target, including sequences of DNA. Certain proteins and peptides bind single-stranded DNA. A number of single-stranded binding proteins exist in nature. In one embodiment, small molecules are bound to the molecule under study. These may remain bound through the course of the experiment; they may be removed during the experiment, for instance upon translocation through the interface; or some combination of these two. The small molecules may be dyes, drugs, or other molecules. They may include the molecules described above, or other molecules.

Molecules that bind in one of the grooves of DNA often employ a mixture of interactions, such as involving both ionic or electrostatic interactions in combination with hydrophobic or van der Waals interactions in combination with hydrogen bonding. Such molecules include the minor groove binding bisbenzimide dyes such as Hoechst 33258 and the antibiotic distamycin-A, as well as major groove binding molecules such as methylene green. In one embodiment, ions or molecules bound to the molecule under study interact specifically with molecules confined to the interface. For example, a molecule bound to DNA might form a stable structure with molecules at the interface, which is then broken as the molecule is moved through the interface, generating a change in force.

### 1.2.7 Polymer Additions to Fluid

Polymers that will interact with the molecule under study or other parts of the system can be added to one or more of the fluids. In one embodiment of the invention, the molecule under study starts in a fluid that has a polymer at a high enough concentration that it is entangled, and the molecule under study entangled with it. The polymer added to the fluid can also be at a lower concentration. It can also be cross-linked, for instance as in a hydrogel. The polymer may be positively or negatively charged, or have a neutral charge, or be nonpolar. It may be a nucleic acid or other biological polymer, or an artificial polymer.

### 1.3 Interface Modification and Engineering

A special case of molecules in one or more of the fluids that associate with, bind to or modify nucleic acids in a way that will produce a signal in the force profile are molecules that preferentially populate the interface between two fluids, as shown in FIG. 1 40. The molecules at the interface may serve as a means to engineer the interface properties, and it may interact with the molecule under study. A common class of surfactants are molecules that have a hydrophobic part and a hydrophilic part and tend to accumulate at air-water or oil-water interfaces by orienting the hydrophobic part of the molecule toward the air or oil and the hydrophilic part toward the water. In the following discussion we use the term surfactant to refer to these molecules, as well as any molecule that accumulates or preferentially associates with a surface or interface between two media. Molecular interfacial layers such those composed of surfactants can be considered as a boundary structure between two fluids. Alternatively, such layers can also be considered and treated as a separate fluid such that a fluid 1 with a surfactant layer (fluid 2) separating it from another fluid 3 is a three fluid or three compartment system or device.

Surfactants modify the interface across which molecules or objects are translocated during a force-based assay. By altering the chemical and physical properties surfactants change the energetics of the crossing of the barrier between the two or more fluids. Surfactants are also positioned to associate with or bind to a nucleic acid molecule as it exits one fluid and/or enters another fluid and thereby modify the force recorded during translocation. Surfactants can be modified or engineered to increase or decrease their binding to different parts of a nucleic acid molecule, including to different bases or parts of bases in a base specific manner. Hydrophilic parts of a surfactant may have attractive interactions with hydrophilic parts of the nucleic acid, such as the sugar-phosphate backbone. Hydrophilic parts of a surfactant may have repulsive interactions with hydrophobic parts of the nucleic acid, such as the bases. Hydrophobic parts of a surfactant may have attractive interactions with hydrophobic parts of the nucleic acid, such as the bases. Hydrophobic parts of a surfactant may have repulsive interactions with hydrophilic parts of the nucleic acid, such as the sugar-phosphate backbone.

One or more of the molecules that bind or associate with nucleic acids can be chemically attached to one a surfactant, restricting the binding molecule to the interface. Restricting molecules that bind or associate with a nucleic acid to a plane or a thin layer at the interface between two fluids can allow for high local concentrations of those molecules, thereby altering the binding kinetics or equilibria with a nucleic acid molecule being translocated across the interface. Molecules at the interface can also contain photoactivatable functions that can modify the structure of chemistry of the interface during translocation.

Common surfactants include the Span family, the Tween family, and Triton X-100, though other surfactants such as the ionic detergents cetyl trimethyl ammonium bromide (CTAB) or sodium dodecyl sulfate (SDS), or any other surfactant may be used. A common class of surfactants are those based on lipid molecules, similar or identical to those found in living organisms. Surfactant layers can be painted, self-assembled, assembled from vesicles or micelles or chemically synthesized in place.

Surfactants can also act as boundaries between two miscible liquids, separating what would otherwise be one fluid into two fluids. For example, a lipid bilayer can be formed such that it separates an aqueous fluid into two parts. Surfactant layers can be partially or completely cross-linked to form a physically more stable layer. Such cross-linking also prevents or reduces the disruption of the surfactant layer by a translocating molecule. Surfactants can also modify evaporation kinetics of solutions.

Surfactants can be combined with applied pressure and the shape and dimensions of an opening to modify the surface tension of an interface. By selecting a combination of parameters or components from those listed above.

Amphiphilic molecules may be biological molecules such as peptides or nucleic acids. For instance, hydrophobins are a class of small proteins that are found naturally in fungi. They can form stable structures at polar-nonpolar interfaces. In one embodiment of the invention, hydrophobins or artificial hydrophobin mutants are present at the interface.

### 1.4 Translocation

A wide variety of mechanisms are available for translocating the molecule through the interface or interfaces, and controlling the applied force on the molecule. Typically, the molecule will traverse the interface along the length of the molecule, and perpendicular, locally, to the interface. However, the molecule may also be translated laterally, meaning parallel to the plane of the interface, or at any angle with respect to the plane of the interface, during the measurement. Techniques for translocation may be combined, for instance, by attaching one manipulator to one end of the sample molecule, and a different manipulator to the other end. This type of double-manipulator strategy may be used to translate the molecule laterally across the interface, and it may be used so that the molecule may be moved in both directions across the interface. We refer to computer control of device elements, which could include personal computers; smartphones; tablets; or standalone electronics, for instance those based on application-specific integrated circuits (ASICs), field-programmable gate arrays (FPGAs); or other computing systems; or some combination thereof. These types of computing systems can also be used for data collection, data processing, data storage, base calling, and user interface.

### 1.4.1 Cantilever Attached to a Nanopositioning Stage

One common method for applying a force to a biological macromolecule is to use a manipulator physically attached to a nanopositioning device, for instance a piezoelectric positioning stage, which is capable of producing precise motion using computer control. Piezoelectric positioning stages are currently commercially available that perform with angstrom or even sub-angstrom resolution. Additional positioning stage technologies capable of angstrom resolution include magnetic voice-coil stages and thermally or electrostatically actuated micro-electrical-mechanical-systems (MEMS) flexure stages. Techniques for controlling and measuring the motion of these stages are known in the art. The transducer in this case can be a MEMS device, for instance a flexure or a cantilever similar to those used in atomic force microscopy (AFM). These can be fabricated by standard lithographic techniques. The transducer can also act as the force sensor. These cantilevers are commonly made from silicon or silicon nitride, and can be made from other materials as well. A wide variety of such cantilevers are commercially available. An array of many cantilevers fabricated into a single chip could be used to probe many molecules simultaneously, and such devices have been previously fabricated. Techniques to move individual cantilevers in an array are known in the art. The cantilever may be driven electrostatically, by applying an external electric field. It may have a coating that responds to a magnetic field, such as a paramagnetic coating, such that it can be driven by an externally applied magnetic field. This magnetic field can be generated by a configuration of permanent magnets, by electromagnetic coils, or a combination of the two. In one implementation, a magnetic field is generated by coils in the Helmholtz configuration. In another, permanent magnets are aligned in a Halbach array, which generates a strong field on one side of the set of magnets. The cantilever can also be fabricated from two materials with different thermal expansion coefficients, so that the cantilever bends when its temperature is changed. The cantilever may also be fabricated from a piezoelectric material which can allow actuation and detection simultaneously.

In one embodiment, the base of a cantilever attached to a nanopositioning stage is moved to translocate the molecule across the interface and changes in the force experienced by the molecule are measured by monitoring the bending at the distal end of the cantilever.

A tip is mounted on the lever or other MEMS device. In one example implementation, the molecule under study is floating free in one fluid (Fluid 1). The tip starts in a different fluid (Fluid 2), is driven across the interface, and attaches, either specifically or non-specifically, to the molecule. The tip is then moved back from Fluid 2 to Fluid 1, leaving the molecule behind across the interface. The molecule is then translocated back across the interface by the tip. In another implementation, the molecules are loaded onto the tip before the measurement begins, for example by dipping the tip into a separate reservoir.

### 1.4.2 Probe Tips

It is often desirable to have a long tip on the end of the cantilever so that the probe, but not the lever itself, traverses the interface during this process. Long tips made of other materials can be used to dip through the layers of fluid and pull the macromolecules back through. These materials can include semiconductors, metals, ceramics, glasses, or polymers. Tips made of semiconductor wires are commercially available, and have been used to measure forces resulting from retracting wires or fibers from aqueous solution into air. Polymers and glasses can be made into long tips by drawing them from a melt, a process commonly used for making glass micropipettes. For example, a micropipette puller is a commercially available device that controls the heating and pull rate to create such a tip. Tips for this purpose can also be made by electron-beam induced deposition of various materials. To do this, a beam of electrons is directed at the location where the tip is desired, for instance, using a scanning electron microscope. A gas is introduced nearby that has a precursor molecule to the desired material, which reacts with the electron beam to form a solid structure. Instruments are commercially available that provide the electron beams as well as a means to introduce the precursor molecule. A wide variety of materials may be deposited in this manner. AFM cantilevers are commercially available with carbon tips grown in this way. Tips may also be milled from existing materials using focused-ion-beam (FIB) instruments which are commercially available.

### 1.4.3 Micropipettes and Microfluidic Manipulation

Fluid flow may be used to manipulate the sample. In one embodiment of the invention, the molecule under study is attached to a bead that is held by a micropipette, held by suction from the other end of the micropipette. Fluid flow, for instance in a microfluidic system, may be used to move and stretch molecules.

### 1.4.4 Optical Tweezers

Optical tweezers, also known as optical traps, are another common technique for applying forces to biological macromolecules. They consist of a bead with a different dielectric constant than the surrounding medium, typically made of glass or a polymer. When a laser beam is tightly focused onto the bead, it produces forces on the bead that hold it in place, essentially at the focal center in all three dimensions. Then the focal point can be moved by moving optical elements to redirect the beam, bringing the bead with it. A molecule under study is attached to the bead, allowing a force to be applied to it. Optical trapping experiments using molecules attached to a fixed surface on one end and the bead on the other end, or to one bead on each end, are common in single-molecule biophysics experiments, particularly those that involve stretching DNA. Techniques for configuring and controlling them are known in the art.

In one embodiment of the invention, a molecule under study is attached to a bead. The bead is then translocated using the optical force from one fluid to the other. This is typically done by moving the bead in a direction perpendicular to the direction of light propagation, as the force is stronger in that direction. After the bead traverses the interface, the molecule under study trails behind, through the interface. In another implementation, one bead begins in Fluid 1 with molecules under study attached to it. Another bead is in Fluid 2. Both beads are brought to the interface using optical tweezers without either bead ever traversing completely across the interface. The non-attached end of the molecule then attaches to the second bead. Optical tweezers have an interesting property that there is a distance from the center of the trap, with the bead near the edge, at which the force is locally independent of distance. The spring constant is effectively very low. This is referred to in the field as a passive force clamp, because it holds the bead at a near-constant force. In another implementation, the bead is situated in a passive force clamp configuration. The bead may also be somewhere between near the center and near the edge of the trap. In one implementation, the bead is pulled through the interface in the regime of the optical tweezers that corresponds to a high stiffness, then the bead is moved to a low-stiffness regime for the measurement, when only the molecule under study is in the interface. In order to study multiple molecules simultaneously, an array of optical traps can be used. These can be generated from a single laser beam by micromirrors or through holographic means such as diffractive optical elements (DOE). Micromirrors can also be used to control the position of each trap. Several beads can also be manipulated in parallel by quickly moving a single trap from bead to bead, moving each in sequence.

While optical tweezers may be formed from macroscopic optical elements including traditional microscope objectives, arrays of tweezers may also be generated by microscopic electro-optic elements such as waveguides, photonic crystals, or divergent field traps, allowing arrays of thousands of traps to be made on an individual chip. The type of polymer or glass beads used in optical trapping may also be manipulated by pressure waves in the liquid.

### 1.4.5 Magnetic Tweezers

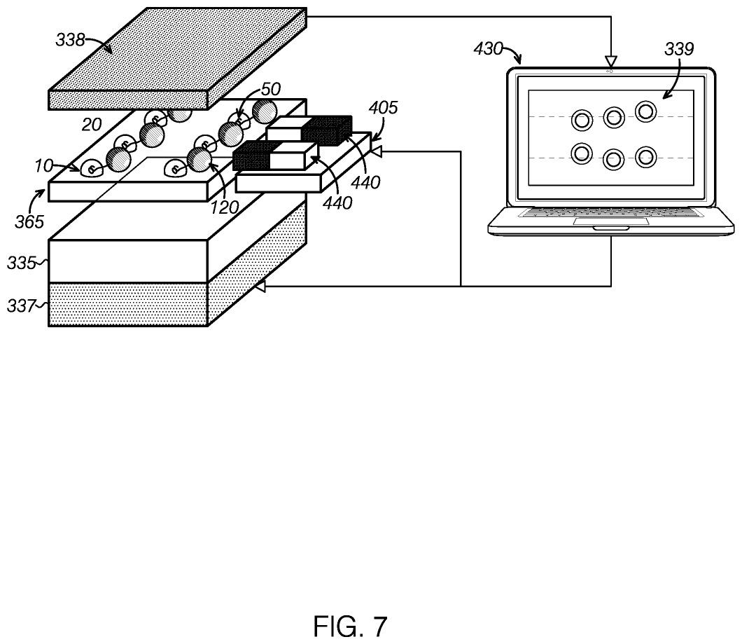

Magnetic tweezers, also known as magnetic traps, are another common technique to apply forces to biological macromolecules. In this technique, molecules are attached to a bead that responds to a magnetic field, for instance, it is paramagnetic, super-paramagnetic, or ferromagnetic. The end of the molecule can then be moved by changing the strength of an applied external magnetic field gradient. An example is shown in FIG. 7. An array of magnetic beads 120 surrounded by Fluid 2 20 are attached to one end of the DNA 50, whose other end is in Fluid 1 10. Magnets 440 are mounted to a motion controller 405 whose motion can be controlled by a computer 430 in order to control the force on the magnetic beads 120, thus controlling the translocation of the DNA 50 through the interface 30.

The applied magnetic field can interact with many magnetic beads at one time, to translocate several molecules at once across an interface. This magnetic field can be generated by a configuration of permanent magnets, by electromagnetic coils, or a combination of the two. In one implementation, a magnetic field is generated by coils in the Helmholtz configuration. In another, permanent magnets are aligned in a Halbach array, which generates a strong field on one side of the set of magnets. In a Halbach array, each magnet is rotated at 90 degrees from the previous one, so that every fourth magnet is facing the same direction. The array may either be linear or cylindrical. The reaction chamber must be designed such that the magnetic field is strong enough to pull the bead through the interface. In one implementation, this force is 100, 200, 500, or 1000 pN. In practice, this means that the design of the chamber allows the set of magnets to be very close to the interface. The distance from each of the fluids to the interface may be less than 1 mm. The Halbach array may be made of micron-sized permanent magnets, each generating a magnetic dipole just above it. Then each bead attached to a molecule will align itself to one spot on the linear array. Such an array can be fabricated by starting with a linear array of wells. With only every fourth well exposed (1, 5, 9, etc.) beads are introduced, aligned to a magnetic field to orient them in the correct direction, then fastened into the wells. Next, the neighboring set of wells (2, 6, 10, etc.) are exposed, aligned at 90 degrees from the first set, and fastened into their wells. The process is repeated for the final two sets of wells. In another implementation, the magnetic field can be generated by a coil wrapped around a sharp core, which generates a large local field. Configurations using permanent magnets are on nanopositioners, for instance piezoelectric stages, to move and control the beads. Magnetic field configurations described here can also be used with the MEMS manipulators described elsewhere.

### 1.4.6 Electrostatic

The DNA may also be manipulated electrostatically, for instance as is done in electrophoresis. To do this, an external voltage is applied across a distance. DNA, being negatively charged, will move toward the positive side. Electrostatic manipulation can similarly be used to move other molecules that are charged. It can also be used to move larger objects, such as MEMS elements or beads, that are charged, and it may be used to manipulate droplets on a chip, as in digital microfluidics. Electrostatic forces can also be applied to particles, such as beads or droplets, that have an overall neutral charge, typically by inducing a dipole. For instance, forces may also be applied to neutrally charged dielectric beads using non-uniform electric fields, a technique known as dielectrophoresis. This can also include dielectrophoresis that is induced optically, using light.

### 1.4.7 Acceleration-Based

The molecule may be manipulated by attaching it to a particle with high inertial mass, then applying an acceleration. For instance, the gravitational force can be used to move the molecule through the interface. A centrifuge can be used to stretch single biological molecules. Such a device can also be used to pull the molecule under study through the interface. In one implementation, the molecule is attached on one end to a particle with high inertial mass, while the interface is arranged to be in a centrifuge. The acceleration from the centrifuge pulls the particle and the attached molecule through the interface. The centrifuge motion can be adjusted so that when the particle is going through the interface, the force is higher, then a lower force is applied during the measurement. Three stages can be employed: one to bring the particles to the surface, one to pull them across, and one for the sensing step. The end of the molecule can be either free or attached to a second particle. The bead can be tracked using a tightly focused beam similar to that used in optical trapping, but with a much lower power so that it is only sensing rather than manipulating the molecule. The centrifuge device can be made from a disc-shaped chamber, so that when it is spun, the more massive fluid is concentrated to the edges of the disc.

### 1.4.8 Translating a Meniscus Across the Molecule

A very different configuration is to immobilize the molecule at one or both ends, then move the interface across it. One example of this has been described previously in the literature as “molecular combing” as a method for depositing and aligning DNA onto a surface. The DNA is bound to the surface, while an interface moves across it, pulling the DNA from aqueous solution into air. This can be done by using a mechanical object to pull the interface along at a desired rate. Another way to configure this is to hold the molecule under study by both ends and move a liquid-liquid interface across it lengthwise, for instance in a microfluidic chamber by pushing and pulling liquid through one of the sides. Another way to achieve the same effect is to attach one end of a molecule to the bottom of a chamber, stretch the other end with a magnetic bead in an externally applied field, and raise and lower the interface to scan it across the molecule. Droplets can also be moved across a bound molecule using a technique known as digital microfluidics. Here, droplets are moved across a surface of a chip by strategically applying electrostatic forces to pads under the droplets. Some of the first measurements of single DNA molecules were made by stretching the molecules using flow in a microfluidic chamber. Such a situation could be engineered, and then a new liquid introduced, so that the interface between the two liquids moved across the molecule. In one implementation, the molecule is held fixed on both ends while the meniscus is arranged so that it is crossing the molecule, then freed from the fixed edges and moved with a manipulator or manipulators. In one implementation, a molecule begins in an aqueous solution that fills a disc-shaped chamber. The molecule is attached to MEMS-based force sensors, for instance cantilevers, surrounding a spindle at the center of the disc. As the disc spins, a lower density fluid is drained from the edges and a higher density fluid is added to the center, so that the interface slowly moves across the molecule.

### 1.4.9 Evaporative Translocation

An alternative approach to translocate a sample relative to an interface is to use evaporation or condensation. Holding a sample attached to a tip in place while allowing the liquid in one chamber to evaporate causes the interface to move, which will produce a change in the force on the sample. Evaporation is achieved by reducing the vapor saturation to below the dew point, by 1%, 2%, 4%, 8%, 16%, 32%, 64%. Condensation into the medium can be achieved by maintaining the vapor of a molecule at a temperature 1, 2, 4, 8, 16, 32 or 64 degrees C. above the dew point, while maintaining the temperature of a medium at the dew point or 1, 2, 4, 8, 16, 32 or 64 degrees C. below the dew point. These conditions will cause the position of the interface between the two media to move relative to the static position of the tip. By controlling the balance of temperature and vapor saturation, the rate of evaporative or condensation-based movement of the interface can be controlled between 1 nm/s and 10,000 nm/s. Changes in force are then detected as described elsewhere herein for the purpose of nucleic acid sequencing and other applications. If this is performed near a surface, as is done in molecular combing, it results in a DNA that is laid flat on the surface, which can then be subjected to further study.

### 1.4.10 Force Control on Both Ends of the Molecule, or Just One

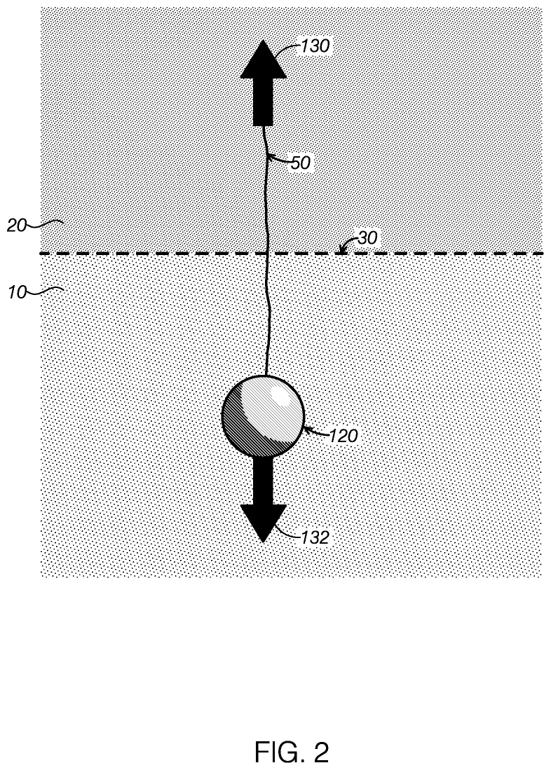

In general, the force on the molecule used to translocate it through the interface can be applied from one or both ends. Each end of the molecule may be attached to a non-moving surface, such as the side or the floor of the reaction chamber; attached to another object that can be moved by the user; or free in solution. Different techniques for moving the molecule ends can be combined. For instance, one end can be held using a MEMS device, such as an AFM cantilever, and the other end moved using magnetic tweezers. FIG. 2 shows a strand of DNA 50 attached on one end to a magnetic bead 120. The DNA 50 is translated across an interface 30 from fluid 1 10 to fluid 2 20. A force 130 is applied to one end of the molecule using any of the various methods described for translocating the molecule while monitoring the force. A magnetic bead on the other end of the molecule is subjected to magnetic field gradient and applies a second force 132 to the molecule. A nucleic acid free in solution will collapse into a ball shape, which provides an anchor preventing the molecule from moving across the interface all at once, since it would require a higher force to move the ball across the interface than a strand unwound from the end of the ball. Holding the second end of the molecule is therefore not required. However, applying a second force can straighten the molecule in Fluid 1, thus ensuring that only a small part of the molecule is interacting with the interface at any given time, increasing the fidelity of the measurement. Attachments can be made and destroyed during the course of the experiment. For instance, a molecule may be attached to a fixed wall while it is originally brought across the interface, then attached to two manipulators during the force measurement step. The force required to move the manipulator through the interface can be much greater than the force required to move the molecule under study through the interface, and by attaching and detaching the molecule, these can be supplied by different means. If the molecule is held by two ends, it may be also be translated laterally along the interface.

### 1.4.11 Attaching the Molecule to the Manipulator

Whatever inorganic surface the molecule is held by—the wall of a chamber, bead, or tip on cantilever—it must be attached to that surface somehow. The bead, tip, other MEMS device, or other device used to move the molecule are referred to as the “manipulator.” In one embodiment, the adhesion step occurs in the same fluid chamber as the experiment, and, in another, the manipulator is loaded with molecules in a separate “load chamber,” then transferred to the chamber that contains the measurement interface. In one embodiment, intermediate steps are used to attach the molecule between the “load chamber” and the measurement chambers, including steps that take place in liquid or gaseous surroundings, or that involve temperature control. For example, the molecules may be baked on to the tip. Double stranded nucleic acids can be attached to the probe by one end, then denatured so that the non-attached end falls away from the manipulator. In one embodiment, molecular attachment to the surface is achieved by molecular combing. Often single molecule force studies simply use non-specific attachment, with unknown mechanism, to attach molecules to surfaces. In one embodiment, nonspecific sticking from a solution is used to attach the molecule to the manipulator. The tip can be made from a polymer that adheres to the molecule. There are a large number of specific molecular attachments commonly used in the field as well. In one embodiment, silane chemistry with a specific functional group is used to attach the molecule to the manipulator. In one embodiment, antibodies are used to make the attachment. In one implementation, aptamers or SOMAmers are used to make the attachment. Attachment may also be performed using functionalized oligonucleotides that have been attached to the nucleic acid during sample preparation. End groups on the nucleic acid may include biotin, primary amines, carboxylic acids, thiols, dithiols. The manipulator may be modified using vapor deposition, electrochemical deposition, adsorption or other means to facilitate molecule attachment. The manipulator may be modified with an alkyne containing molecule. The manipulator may be modified with a Phos-Tag (1,3-bis[bis(pyridin-2-ylmethyl) amino]propan-2-olato dizinc(II) complex) to facilitate attachment of the molecule via phosphate at the end of the molecule. Attachments are chosen to be stable in both solutions. The manipulator can be functionalized, then the non-functionalized part coated with a type of molecule that does not stick well to the molecule under study, in a process known as passivation. Poly-ethylene gycol (PEG) can be used as a passivating molecule. This passivation can be done as a second step in the manipulator functionalization or at the same time. In one embodiment, a glass or semiconductor manipulator surface is coated with molecules containing a thiol group, which reacts to form a covalent bond with a functional group added to the molecule under study. The manipulator can also be made from a material that contains thiol groups without an additional coating. The manipulator can also be made of gold, or coated in gold, and react with a thiol group on the molecule. In one embodiment, a thiol group on the molecule forms a bond with a maleimide group on the bead. In one embodiment of the invention, multiple polymers are attached to the manipulator, and interactions between them generate measurable forces. Catch bonds are formed from binding partners found in nature where the strength of binding increases upon applied force. In one embodiment, natural or engineered catch bonds, are used in the attachment. Such an attachment would allow a molecule to remain attached to the tip while it is under tension, but the molecule could be released by lowering the tension. This would provide a facile mechanism to exchange molecules on the tip.

### 1.4.12 Detecting Attachment Events

In some embodiments, the tip begins with no molecule attached, then is translocated across the interface and a molecule attaches to it, for instance through non-specific adsorption. In such cases, the conditions may be such that many attempts to attach a molecule are unsuccessful. These conditions can be tuned to achieve a desired fraction of events that are successful in attaching a molecule. Conditions can be tuned so that attempts resulting in more than one molecule attached to the tip are very rare. In this and other configurations, it can be desirable to detect whether a single molecule is attached to the tip (an “attachment event”), then perform further study on that molecule. This can, for instance, increase efficiency and throughput by decreasing time spent performing translation and force measurement protocols when no molecule is attached. Fouling can also be detected so that fouled tips can be discarded or cleaned.

Several methods are available for detecting attachment events. An attached molecule exerts a force that can be distinguishable from a situation where no molecule is attached, several molecules are attached, or the probe is completely fouled. Although the force signature depends on the sequence, that signature is on top of a plateau force that is different than when no molecule is attached. For the same type of molecule, the plateau force will be within a range. Attachment events can be detected by observing the force plateau and sensing when it comes within the desired range. An attempt to attach a molecule can proceed as follows: First, translocate the tip from Fluid 2 into Fluid 1, where the target molecule is present in Fluid 1 but not Fluid 2. Second, pause to allow the molecule to attach to the probe tip. Third, pull the tip back through the interface. Fourth, once the tip is at the desired height above the interface, begin the attachment sensing routine. If the force at that point is less than the desired range, interpret it as an event with no molecule, and make another attempt. If the force is greater than the desired range, interpret it as multiple molecules attached, and continue to extract the molecule. If the force reaches the desired range, interpret it as a single molecule attached, and continue to further study. This could include proceeding to a pre-determined automated or semi-automated routine, for instance, the flossing routine described below; or, it could include waiting for further user input. In an array of tips, tips with molecules attached can be made to wait while other tips in the array make more attempts to attach molecules, repeated until a desired fraction of tips in the array are attached to good molecules. Fouled tips can be removed from further analysis, and flagged to the user. A fouled tip could also be subjected to a cleaning regimen, for instance by inserting it into a cleaning solution or removing the fouling material electrostatically. The baseline force can be determined from a previous force curve event, or from the force measured on the ingoing part of the curve. Other ways for detecting an attached molecule include comparing the standard deviation of the force profile with that of an unattached profile; comparing the number of step-edge features; comparing the skew of the curve or its derivative. It might include using the base calling algorithm in real time to attempt to sequence a section, and concluding that a molecule is attached when the sequencing is successful. Methods external to the force measurement, such as detection of longitudinal current along the molecule, can also be used to detect molecular attachment.

After detecting an attachment event, the tip may be lowered very near to the interface without touching it again, which can help study the portion of the molecule that is nearest to the tip.

### 1.4.13 Modifying Probe to Minimize Entry and Exit Force Through Interface

Large forces can occur when relatively large objects (micron-scale) are translocated through an interface, which can obscure the desired force signal from the molecule. These forces can also make it difficult to measure the portion of the molecule closest to the attachment point to the transducer. The contact angle of the fluids with the object and the shape of the object will determine the forces experienced by the object while it is in contact with the interface. If the shape of the object maintains a constant angle relative to the interface, for instance it is shaped like a cylinder or cone with its axis of symmetry perpendicular to the interface, its contact angle with the interface can be matched to that angle so that the interface remains locally flat, thus minimizing or eliminating the forces experienced by the object at the interface. For example, a cylinder with a contact angle of 90 degrees experiences no force while it is piercing the interface. Similarly, a cone where the contact angle is 90 degrees plus the half angle of the cone will experience no force as it pierces and moves through the interface. This technique is not available to techniques based on spherical beads, as the angle between the bead and the interface changes as the bead is pulled through. For a polymer tip, choice of polymer can set the contact angle. For instance, the common polymers polypropylene, polyethlyene, and polystyrene typically have contact angles at the air-water interface of 102.1, 96, and 87.4 degrees, respectively. In one implementation, the tip is made from a polymer whose contact angle at the interface is chosen to be 90 degrees plus the half angle of the cone. In another implementation, the tip is made from a polymer whose contact angle is selected to be mismatched by a particular value to create an attractive or repulsive force. Changes in contact angle can be affected by functionalizing the surface. In one implementation, the surface of the manipulator is modified so that its contact angle matches the angle at which the probe surface interacts with the interface. In another implementation, the surface of the manipulator is modified to be mismatched from the angle of the interface by a particular value. One common method for functionalizing glass or silicon surface is using silane or cholorosilane chemistry. Silanes and cholorosilanes are molecules that bind covalently to glass or silicon and are commercially available having a wide range of functional groups. In one implementation, the modifications above are achieved through silane chemistry.

### 1.4.14 Repeatedly Translocating the Same Molecule Across an Interface

Whatever the means of translocating the molecule across the interface(s), it can be advantageous to repeatedly translocate the same molecule across the interface in a “flossing” motion to repeatedly measure the force profile of the molecule. This flossing motion can take place across subsections of the molecule that are any desired length. The force can be recorded in either or both directions during the flossing motion and repeated force profile measurements (“floss events”) can be averaged to increase signal to noise in the measurement. Flossing data series could contain tens, hundreds, thousands, ten-thousands, or more floss events. Alternatively, the sequence of the DNA molecule can be determined from each separate floss event and then the majority base at each called position can be used. Each floss event in an averaging set can be made at the same pulling speed, or they may be made at different pulling speeds. Individual floss events in a data set could be the same length, or they could be different length. For instance, each floss event could be longer than the previous one. The position series over time could be input before the flossing occurs, could be determined algorithmically as the measurement proceeds, or could be adjusted by the user as the flossing proceeds. The flossing can be made to wait for user input at the beginning of the flossing event, at pre-determined points during the flossing, or at points in the flossing that are algorithmically determined during the measurement. For instance, the flossing can determine the signal-to-noise that has been achieved, and wait for the user to decide whether that is sufficient, or whether more flossing should occur.

Algorithms may be used to align individual floss events prior to averaging. For instance, an algorithm may find a portion of the trace with a step edge that occurs in all floss events, and align them to that. The DNA may be marked in order to facilitate this process. For instance, a protein may be made to bind to the DNA as a marker that would require a higher force to pull through the droplet, then that higher force could be used as a marker to align floss events. In other polymers such as proteins, structured regions could be used as markers. Alignment for averaging could involve stretching curves, as well. For instance, the algorithm known as Dynamic Time Warping (DTW), used in voice recognition, could also be used here to align curves to each other.

Flossing can also be used to increase the fidelity of the measurement in a particular portion of the molecule being sequenced. Many genetic diseases are the result of a change in a single base at a particular position in the DNA known as a single nucleotide polymorphism (SNP). The sequence of the DNA molecule being measured can be determined in real time from the force profile and when the portion of the molecule where the SNP is located is close to the interface, the molecule can be repeatedly flossed around that position to determine the sequence in the vicinity of the SNP with high fidelity. An artificial intelligence agent, for instance a software agent trained using reinforcement learning, or other machine learning based technique, may be used to determine the optimal sequence and timing of the flossing. This may be done ahead of the experiment or in real time as the measurement progresses.

The polymer can be pulled back across the interface by interfacial forces themselves, or it can be subjected to another force by a manipulator on the other end.

Similarly to the attachment detection routines described above, an event where the molecule detaches from the tip during the flossing or any other process can be detected. This can appear in the force profile as a sudden decrease in force. That tip can then begin again to measure a new molecule.

The methods described for nucleic acids and polymers here includes other types of polymeric molecules and can also be applied to other types of objects. Flossing can be performed with any polymeric molecule such as a polynucleotide, protein, polysaccharide, polyethylene glycol, polyvinyalchol, block co-polymers, etc, or any structure or object that can be translocated across an interface. Other objects include beads, particles, membranes, fibers, blocks of any material that is not soluble in either or any of the liquids used to form an interface. The objects can be of arbitrarily dimensions, limited by the ability to measure forces associated with the translocation to sufficient accuracy and precision.

Direct sequencing of proteins is a measurement of particular interest for many applications, but brings additional challenges over sequencing of DNA. Proteins are made from a library of 20 (encoded) or more amino acids, rather than the four bases for DNA. The chemical differences between many of the amino acids are small, leading to small force signature differences. In addition, the distance between amino acids in a protein is smaller than between nucleobases in a nucleic acid. Flossing will allow a higher signal-to-noise ratio for force curves, improving call accuracy in protein sequencing.

The flossing motion can be a simple sinusoid or saw tooth pattern, or it can be a more complex preprogrammed pattern or a dynamically adaptive pattern. That pattern can be a combination of several other simple patterns. For example, a 3 nm period sawtooth, can be combined with a 30 nm saw tooth and a 300 nm saw tooth. The flossing pattern can also include a randomized component to the translocation or application of force that would reduce or eliminate undesirable contributions from period motions, such mechanical resonances in the measurement system, molecule, liquid or interface. The flossing pattern includes not only patterns in distance or length traversed, but also patterns in speed, velocity or acceleration of translocation. The velocity can follow programmed patterns or mathematical functions, or it can be adaptive and adjust to performance parameters of the system or the detection of specific sequences or sequence characteristic. It can be desirable to floss more quickly at the beginning of a molecule, when the length of the molecule out of solution is shorter. Such a molecule is typically stiffer than a longer molecule, while a longer and more elastic molecule may produce better sequence when translocated more slowly. The flossing pattern can be established using a combination of known sequences and machine learning tools, to establish optimal translocation dynamics. The Flossing pattern can also include or be combined with lateral motions of the polymer being flossed relative to the interface (i.e. in the plane of the interface). Thus the molecule can be flossed at varying angles relative to the interface from 0 degrees (parallel to the interface) to 90 degrees (normal to the interface, or vertical). The flossing pattern may also include a combination of lateral and vertical motions.

### 1.5 Force Measurement

### 1.5.1 Microfabricated Force Sensor

Physical force sensors can be fabricated using a variety of lithographic techniques known in the art, including but not limited to sputtering, deposition by evaporation, shadow masking, photolithography, electron beam lithography, and direct patterning. Many such fabrication steps can be combined to build a force sensing device. Microfabrication techniques may be used on a wide variety of materials, including but not limited to silicon, silicon nitride, gallium arsenide; metals; ceramics; photoresists such as SU-8 and other polymeric materials. Devices may be made of one, two or more materials. These force sensors can have a variety of physical shapes, including but not limited to crossbars, cantilevers, and membranes. These microfabricated features can be fabricated to act as springs, linear over a certain deflection range, with a large range of spring constant. Their force-distance response may also be nonlinear over the relevant range. The force-distance response of the device may be calibrated ahead of time. AFM cantilevers are one class of commercially available microfabricated devices. For instance, spring constants for AFM cantilevers may be used in the range of 0.005 to 100 N/m. A wide range of techniques exist for detecting the deflection, and therefore, once the spring constant is known, the force, on an AFM cantilever. The most common, typically employed on commercial AFM is known as the optical lever technique. In one embodiment of the invention, the optical lever technique is employed, either on a cantilever (supported on one side) or membrane (supported on all sides), on a crossbar (supported on two sides), or on another structure. Parallel optical levers may be made to track an array of tips, including splitting the beam using diffraction, illuminating the entire array, time-sharing the beam across the cantilevers, or by other means. Once many laser beams are present, each beam can be steered, for instance by using a micromirror array. A cantilever or membrane may also be made to sense the force internally. For instance, a current path fabricated on the force sensor can be fabricated to act as a strain gauge. Deformation of the element due to bending of the lever, membrane, or other sensor causes a change in resistance, i.e. piezoresistance, which can be detected and quantified. A cantilever may have a simple rectangle shape, or it may be a more complicated shape. For instance, cantilevers with thinned or cut out regions may be used. The cantilever may be fabricated, then modified, for instance by focused ion beam milling. The observation of bending may be made at low frequency, significantly below the first resonant frequency of the device; on any resonant frequency of the device, including torsional and lateral modes; or above the resonant frequency of the device.

The thermal motion of the microfabricated force sensor itself can be a significant noise source impeding the measurement. One way to decrease the effect of this noise is by making the force sensor smaller. While the magnitude of the thermal motion is determined by the stiffness of the sensor, making the sensor smaller increases the resonance frequencies of the sensor and spreads the thermal motion over a much larger frequency range, resulting in less noise per unit bandwidth. Miniaturization of the sensor also improves the piezoresistive detection technique described above because for a given deflection of a force sensor, the strains generated are higher when the object is smaller because the local radii of curvature are proportionately smaller. Accordingly it is advantageous to make the size of the microfabricated force sensor as small as possible. The microfabricated force sensor may be a cantilever where the largest dimension is smaller than 100 μm, 10 μm, 5 μm, or 1 μm.

### 1.5.2 Camera-Based Bead Tracking

Any bead-based technique can be sensed using optical tracking. In this technique, the light scattered from the beads is imaged onto a camera, which includes a sensor chip as well as an associated optical system and related electronics. This allows many particles to be detected simultaneously. Then the signal from the detection chip is digitized. At each camera image frame, the position of each particle can be determined, typically by fitting the peak of the Airy disk using software. The trajectory of the bead can then be determined. The spring constant of the traps can be determined ahead of time, and from this the force on the bead calculated from its position. A graphics processing unit (GPU) may be used to perform these calculations quickly. An example is shown in FIG. 7. In this device, an array of magnetic beads 120 are illuminated from one side by a widefield light source 335, which can be controlled by a computer 430 through a controller 337. Light passes through the slide 365 supporting the array, scatters from the beads 120, and lands on a camera 338. The image 339 is then recorded using the computer 339. Displacement from equilibrium generated by the force may be used as the signal, it may be converted into force using calibrations, determined before, during, or after the experiment, or the images may be used raw, without extracting distance or force from them. Smaller sections of image data may be used, for instance regions of interest or line scans.

### 1.5.3 Scattering-Based Bead Tracking

Bead position in an optical or magnetic trap can also be detected through scattering of a tightly focused laser beam. The three-dimensional position of asymmetrical objects such as AFM tips can also be measured in this way. An optical tweezer geometry provides a natural way to sense bead displacement in three dimensions, as it contains a tightly focused beam. The trapping beam itself can be used to detect position of a bead, or another, weaker detection beam can be introduced. Light scattered from the bead is collected onto a position-sensitive detector, such as a split photodiode or position-sensitive photodiode array. The position is read out by using low-noise amplifiers. The signal may then be digitized. The focused laser spot may be stationary with respect to the sample or it may follow the bead using a mirror, or, for a multiplexed system, with a micromirror array. Before the experiment begins, the spring constant of the bead/trap system may be calibrated using one of a few techniques known in the art, such as monitoring the thermal motion of the bead. The force may then be calculated from the position.

### 1.5.4 Measurement of the Interface

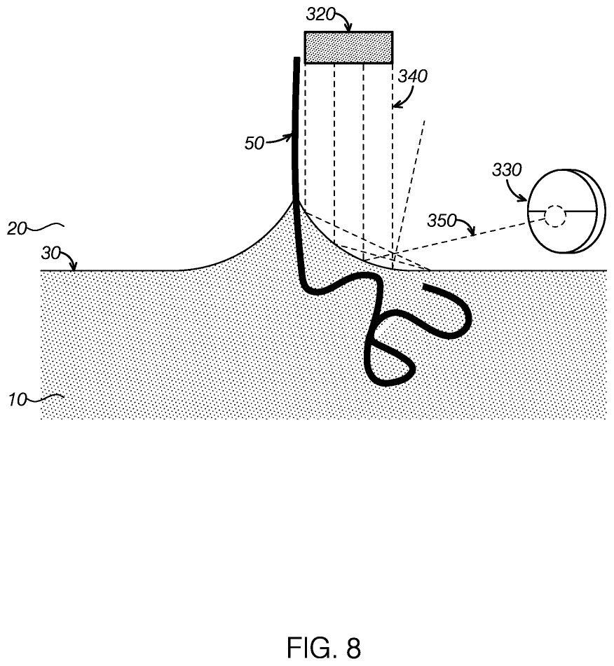

During translocation of the molecule under study through the interface, the interface itself will locally deflect differently depending on the force applied to it. This deflection can be measured by scattering a laser beam from the interface and collecting the scattered light. This light would then be collected by a position-sensitive photodetector, such as a split photodiode, a position sensitive detector based on PIN photodiode technology but without discrete elements, a camera chip, or other position-sensitive detector. A camera chip or other array of photodetectors could be used to detect multiple signals at once. This is very similar to a technique known as photothermal displacement spectroscopy, but here used on a liquid rather than a solid surface. Photothermal displacement spectroscopy is capable of measuring deflections of a surface to 10⁻⁴ Angstroms. Nano or microparticles can be added to the interface to increase scattering by increasing the reflectivity of the interface. The two fluids can be selected to maximize the difference in index of refraction, thereby maximizing the scattered light. FIG. 8 shows an example of sensing the curvature of the interface. As the DNA 50 is passed from Fluid 1 10 to Fluid 2 20, it causes the interface 30 to curve. A light source 320 is positioned above the interface 30 so that its incident beam 340 interacts with the curved part of the interface 30. As the curvature of the interface 30 changes, the reflected light beam 350 will change in turn. These changes can be detected on a position sensitive photodetector 330. Alternatively, vibrations of droplet can be introduced, for example, by piezoeletric or other acoustic elements and the effect of the molecule on oscillations of the interface can be measured.

Total internal reflection is a natural phenomenon whereby, when going from a material with a higher index of refraction to one with a lower index of refraction, above a particular angle of incidence, the propagating electromagnetic field does not cross the interface. Just below this critical angle, the reflection and transmission are strongly dependent on the angle of incidence. To measure deflection of the interface, the light can be introduced in this regime, and either the reflected or transmitted light measured. Deflection of the interface will cause a different amount of light to be reflected and transmitted, and this can be measured with a photodetector and digitized.

Total internal reflection also causes modes inside spheres and other shapes known as whispering gallery modes that have mode shapes with all the electromagnetic energy located close to the surface. Whispering gallery modes can be excited in a droplet and these modes are highly sensitive to changes at the interface, resulting in a small change in wavelength of the mode which can be monitored using, for example, a laser to excite the mode and a spectrometer to determine the wavelength of light exiting the droplet. Alternatively, a filter could change the wavelenghth shift into an intensity shift that can be monitored by a photodiode. Such a system could detect changes in the mode wavelength as the molecule translocates the interface.

Interface motion may also be detected using a widefield optical technique. Light passing through the interface will be scattered differently depending on the curvature of the interface. Changes to the interface curvature near the molecule during translocation of the molecule will, in turn, change this scattering. The light pattern produced by the scattering can then be detected using a camera, along with the proper optical system. Effects on the curvature of an array of molecules passing through one or more interfaces can be recorded into an image in this way.

### 1.5.5 Force Sensor on Handle

A force sensor can be engineered into nucleic acids that serve as the handle for the molecule under study. The handles may be made of nucleic acids, proteins, other biological macromolecules, synthetic polymers, other materials, or any combination of these. In one embodiment, the force sensor is a nucleic acid hairpin whose stability depends on the force. At higher force, the hairpin opens, and at lower force, it closes. Since the force changes as the bases traverse the interface, the opening and closing of the hairpin will change in turn. In one embodiment, other configurations are used where two points on the handle are closer or farther apart depending on the force on the molecule. Many of these hairpins or other force-dependent sensors could be employed in series, with each having a slightly different opening force. Then the number of open hairpins can be counted to determine the force: one opened hairpin would indicate a smaller force, while several would indicate a greater force. In one embodiment, fluorophores are engineered into the force detection sections. These fluorophores may be chosen to interact with each other through Foerster resonance energy transfer (FRET), a known method for detecting distances in biophysical experiments. Fluorophore A is excited by an external beam of light. If it is close enough to Fluorophore B, it transfers the excitation energy and Fluorophore B fluoresces. If it is too far away, the energy is not transferred and Fluorophore A fluoresces. The fluorophores emit light at different colors, so it is possible to tell which one is fluorescing at a given time. A wide variety of fluorophore pairs that work well in this configuration are known in the art, and many are commercially available. The characteristic length over which this happens is typically in the range of a few am. The arrangement and opening strength of the hairpin or other force-dependent molecular arrangement can be chosen to maximize the signal at the required force. Other detection techniques could be used to detect molecular re-arrangement of engineered force sensors in the handles. For instance, colloidal particles will scatter light differently when they are near each other than when they are far from each other. The molecular arrangement hosting the FRET pair can also be engineered to have a non-linear spring constant in the direction of force. While the total force required to translocate the molecule across the interface may be of the order a few hundred picoNewtons, the changes in force resulting from different bases translocating the interface may only be a few picoNetwons. By engineering a non-linear spring constant, the FRET contrast can be enhanced in the force region where the base signatures lie.

1.5.6 Detecting Interface Re-Arrangement with FRET

Not only molecular handles rearrange upon force changes, but the interface itself is rearranged. Molecules or colloidal particles can be added to the interface that are tagged with fluorophores. When the interface is rearranged by the molecule under study being pulled through it, the fluorophores move closer together or farther apart. For instance, a collection of amphiphilic colloidal particles can be introduced at an oil-water interface that aggregate near the molecule being measured as it transverses the interface, forming a “pore on demand.” These particles can be engineered to self-assemble so that one of each of a FRET pair is present at the point where the molecule transverses the interface. As the molecule traverses the interface, this “pore on demand” will change shape, moving the particles closer and farther apart, affecting the FRET signal. The FRET signal can then be read using a standard microscope setup. A large number of these signals can be read in parallel. Amphiphilic fluorescent probes may also be introduced at the interface, and their rearrangement sensed as the molecule transverses the interface.

### 1.5.7 Acoustic/Pressure Waves

Pressure waves in each fluid will be generated by the DNA translocating through the interface. These will propagate and may be sensed, for instance by MEMS-type sensing features, at some distance from the molecule.

### 1.5.8 Combined and Hybrid Detection

Force based measurements of chemical and physical properties of molecules or objects being translocated across an interface can be combined with other types of measurement, either simultaneously, in parallel or in series. The combination of different modalities of detection increases the information gathered and thereby allows for faster, more accurate, more complete and more useful information about the sample and its properties to be gathered. In the preferred embodiment force-based detection is combined with electrical detection, where electrical properties such as current flow, voltage, resistance or impedance (or some combination thereof) between the tip in one medium and another medium connected via a nucleic acid molecule. This provides an electrical signal with information that partially, mostly or completely independent of the force measurement. A nucleic acid molecule attached to a tip is translocated across an interface from one medium to another, and a voltage is applied between the tip and the medium from which the molecule is being translocated. As electrons move between the tip and medium via the nucleic acid (or molecules directly associated with the nucleic acid). The flow of the electrons and other electrical properties will depend on the organization of atoms and molecules where the nucleic acid chain exits one medium and enters the second. The flow of electrons will depend on the base transiting the interface, the orientation of the base transiting the interface, the molecules in the first medium and the second medium as well as the molecules at the interface. Alternatively force based detection is combined with an optical measurement performed at the point where the nucleic acid transits from one medium to the other. The optical measurement can consist of a scattering measurement due to deformations or other changes at the interface between the two mediums, a fluorescence measurement due to differences in fluorescence of a base in the two media, the deflection of light striking the interface near the transiting molecule in a way that reflects deformation of the interface.

Force-based detection can also be combined with electrical measurements made between two or more points at or near an opening where an interface is formed.

### 1.5.9 Lock-in Detection

Independent of the methods used to translocate the molecule across the interface and detect the force, an improvement in signal-to-noise in the measurement can be achieved by using lock-in detection. A small modulation on the translocation of the molecule is introduced at a fixed frequency or multiple frequencies. This modulation is on the order of the size of the signal to be detected. For sequencing applications this would be the size of a single base. The modulation can be produced by the same method which is used for translocating the molecule or by an additional actuator. This introduces a single frequency component in the force that is related to the local derivative of the force vs distance profile. This signal can be detected using phase coherent detection such as that in a lock-in amplifier at a significantly higher signal-to-noise ratio than the original force signal.

The frequency of modulation can be chosen to be at a frequency where the force noise is lowest. The frequency could be a resonant frequency of the system, above the resonant frequency, or below the resonant frequency. The frequency can be changed during the course of the experiment, or the frequency could be fixed. The lock-in amplification can be implemented in analog hardware, digitally in hardware, including on a reprogrammable hardware chip (e.g. FPGA), or using software.

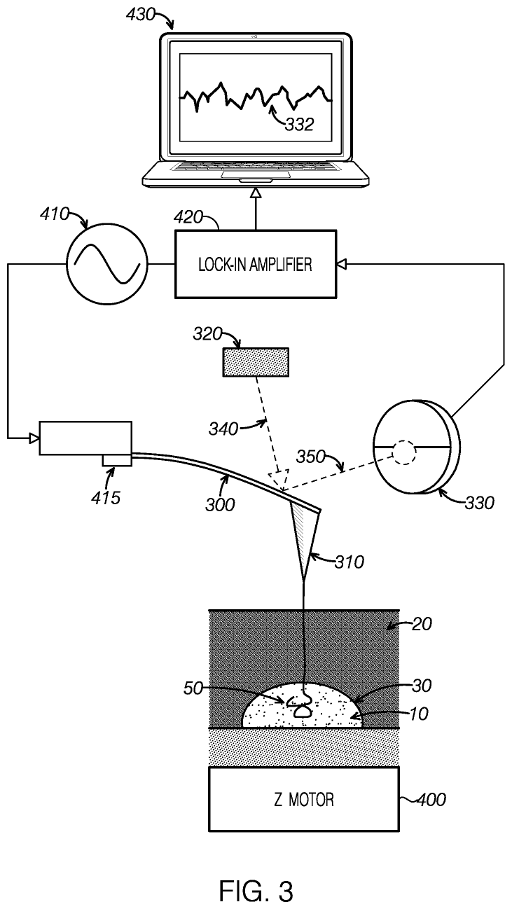

FIG. 3 shows an example of using lock-in amplification in force based sequencing of a DNA molecule using a microfabricated cantilever. A cantilever 300 with a sharp tip 310 is attached to a DNA molecule 50 which is being translocated across and interface 30 between fluid 1 10 and fluid 2 20 by a piezoelectric Z motor 400. An additional dither piezo 415 provides a modulation of the translocation at a frequency produced by the lock-in amplifier 420 or a separate function generator 410. A light source 320 bounces a light beam 340 off the cantilever 300 and the reflected beam 350 is detected by a position sensitive detector 330. The signal from the position detector is fed to the lock-in amplifier 420 and the signal is passed to a computer 430. The computer is used to control the Z motion of the Z motor 400 and generates time series data 332 from the signal received from the lock-in amplifier. The computer 430 is also be used to determine a sequence of bases from the time series data 332.

While this specific example utilitizes a single AFM cantilever and piezoelectric translator, it is understood that the lock-in technique can be advantageously applied in any of the many measurement configurations, including arrays with many detection stations, mentioned elsewhere in the specification.

### 1.6 Examples of Devices

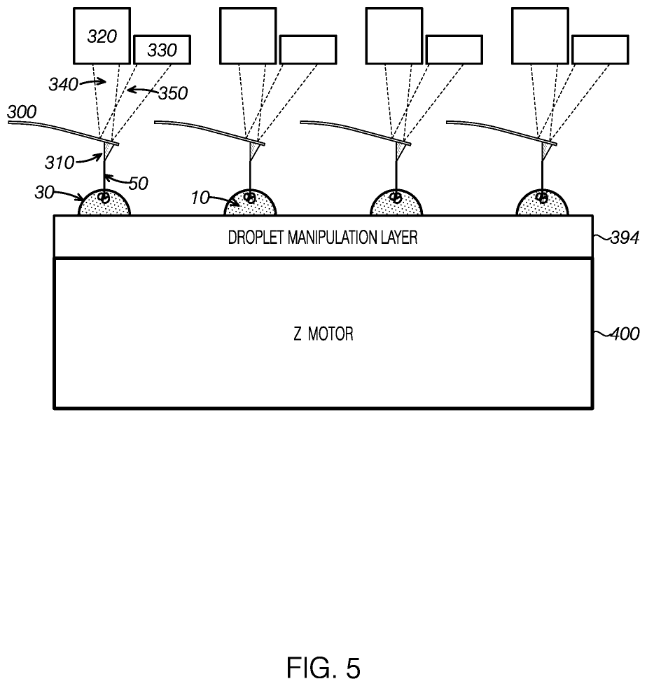

The variety of schemes for translocating the molecule across an interface, measuring the force, and manipulating the liquids can be combined in a large number of configurations to create a measurement device. A few different examples are showing in FIGS. 5, 6, and 7.

FIG. 5 shows an array of cantilevers 300 with tips 310 translocating a large number of DNA strands 50 from an array of droplets 10. A Z motor 400 creates relative motion between the droplets 10 and the cantilevers 300, translocating the individual molecules through their respective interfaces 30. A separate light source 320 for each cantilever directs an input beam 340 to each cantilever and the reflected light 350 is collected by a position sensitive detector 330 which generates a signal indicative of the bending of each cantilever 300. The droplets 10 are arranged on a droplet manipulation layer 394 which may be an array of electrodes capable of moving, splitting, and merging drops by electrowetting. In the case where the fluid the cantilevers are immersed in is a liquid rather than a gas, a separate window (not shown) may separate the cantilevers 300 from the light source 320 and position sensitive detector 330. This window may be a transparent electrode, such as indium tin oxide.

The droplet manipulation layer allows the sample to be pipetted into a loading reservoir (not shown) and the sample can be diluted to a concentration such that droplets split off from the loading reservoir may contain a single molecule on average. These droplets can be transported using the droplet manipulation layer 394 to their individual measurement stations under the cantilevers. The Z motor 400 translates the droplets until they make contact with the cantilever tips 310 and DNA molecules 50 in the droplets become attached to the probe tips either through non-specific binding or specific chemistry. The Z motor 400 then lowers the array of droplets and the forces measures as each molecule translocates its interface is recorded from the position sensitive detectors 330. This can be repeated several times.

Separate electronics and a computer records the data from each position sensitive detector 330 in the array and generates a force profile for the molecule from which the sequence of bases in that molecule can be determined.

To regenerate the array to accept a new sample, droplets with cleaning solutions may be distributed lie under each cantilever 300 and the probe tips 310 may be immersed in the droplets to remove DNA bound to the probe tips. At that point a new sample can be introduced, and the measurement process can start again.

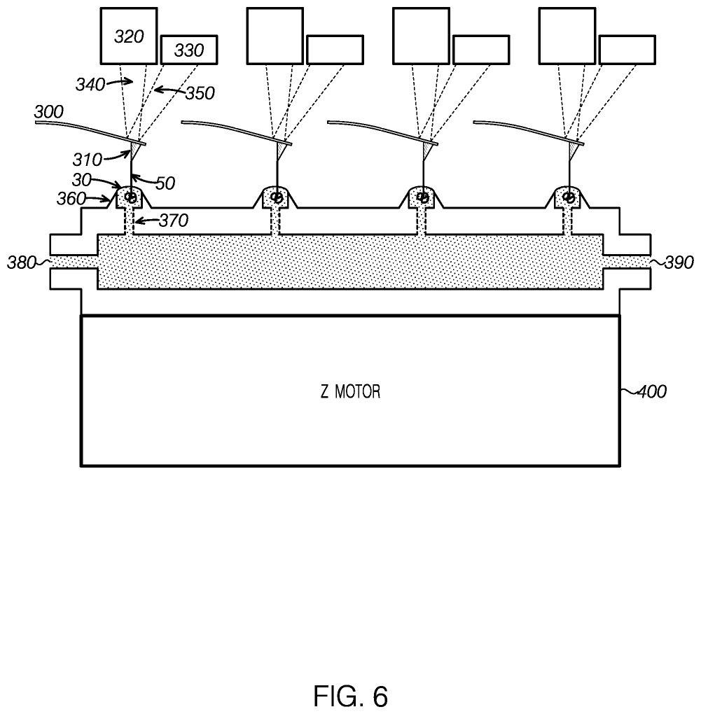

FIG. 6 shows an array of cantilevers with the same detection elements as in FIG. 5, but rather than a droplet manipulation layer, a microfluidic system with a common reservoir with inlet 380 and outlet 390 ports and an array of openings 370 and droplet containment structures 360 is used to create the array of interfaces 30 through which the molecules 50 are translocated. Otherwise, operation of the device is as described above, with the exception of the regeneration step where a cleaning solution can be introduced into the common reservoir to clean DNA off the probe tips 310 to ready the system for a new sample.

FIG. 7 shows an FBS device consisting of an array of droplets of Fluid 1 10 and magnetic beads 120. The droplets are formed in a second fluid, Fluid 2 20 and are supported on a support slide 365. Magnets 440 on a motion controller 405 create a magnetic field gradient that applies a force to the magnetic beads 120. By moving the motion controller 405, the magnetic beads 120 can be driven to the interface with Fluid 1 where DNA molecules 50 can bind to the beads, The motion controller 405 can then be used to move the beads away from the interfaces, translocating the molecules through the interfaces. A widefield light source 335, controlled by a controller 337 illuminates the beads casting an image (which may be formed by additional optics not shown) onto a camera. A computer 430 can be used to calculate bead positions from the camera image 339 as described above. Forces can then be calculated from the measured positions and the base sequence of the molecules can be determined.

It is also possible to replace the support slide 365 with a droplet manipulation layer 394 as described above. In the case the droplet manipulation layer is composed of electrodes to move the droplets via electrowetting it may not be transparent. In that case, the widefield light source 335 and camera 338 may be located on the same side of the device and may view the beads and droplets through a transparent window which may also comprise an electrode made from a transparent material.

### 1.7 Data Collection and Interpretation

### 1.7.1 Recording and Storage of Measurement Data

Measurements from the force sensor and/or other measurement devices may be recorded in a digital form on a computer hard disk, a magnetic tape, a solid-state memory device or other commonly used data storage system. Measurements may also be output to a video display device or to an analog recording device. Data maybe be analyzed in real-time using computer programs or specially designed hardware. Data may be recorded, stored, viewed, or analyzed on a personal computer, smartphone, tablet, purpose-built computing system, or other computing device. Data may be stored or archived for later analysis. Data may comprise raw time series measurements, outputs from lock-in amplification, raw images, or data that has been processed.

### 1.7.2 Form of Time-Series Data

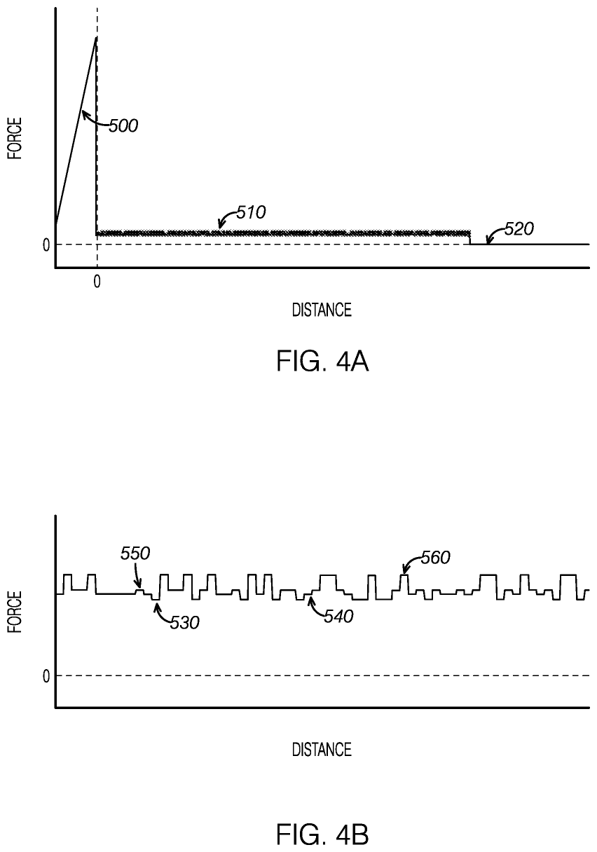

The raw data collected during an FBS measurement may be one or more voltage records collected as a function of time, or it may be two-dimensional data, as from a camera. Camera data can then be analyzed to produce position as a function time, which can be converted to force as a function of time, for instance, by multiplying by a known spring constant for the force measurement system. A FBS force record can be a series of force levels, each level corresponding to a base or short sequence of bases. This example situation is illustrated in FIGS. 4A and 4B. As it comes through the interface, the probe produces a force 500. Then, as the molecule comes through the interface, the force may be plateau-like 510 until it detaches 520. The plateau comprises a series of different force levels 530-560, corresponding to the four bases. It can also be a step-like series of plateaus, where each is characterized by a height and a length. It can also be a more complicated wave form, containing features such as ramps and steps. Each feature in the force record can correspond to a single base, or to two, three, or more bases depending. Force record features and sections can be parameterized in terms of the force magnitude, first derivative, second derivative, and higher order derivatives. Each force signature can also be decomposed into a linear combination of elements. Each force signature can also be examined in frequency space, for instance by applying a Fourier transform. Each force signature could also be characterized by a fit to a polynomial. The signatures can also be parameterized using a combination of these techniques, other techniques, or used as time series or as a function of position without further parameterization.

### 1.7.3 Improving Signal Quality

In some embodiments, one or more techniques may be used to improve the signal-to-noise ratio of a measurement. Any random noise will decrease as more points are averaged, leaving the signal to noise ratio larger. One way to average away noise is to simply wait at a certain point. In some embodiments, one or more pauses are introduced as the molecule transverses the interface, and data is collected for some period of time during the pause. Another technique is to pull the molecule through the interface many times (“flossing”), introducing multiple partial force profiles for the molecule, each comprising a subset of the entire molecular force profile. Partial force profiles acquired over the same sections of molecule may be averaged.

A force balance mode can be used to reject large offset forces while maintaining the ability to detect small signal forces. In this mode, the interface and/or the fluids are tuned so that the average force at the interface is near zero. Then, any deviation due to changes in the molecule as it transverses the interface will be the signal corresponding to different elements being read. Adding amphiphilic molecules, such as lipids or surfactants, to the interface can be used to control the surface tension, as can lateral pressure on the interface.

In an array of force manipulators and force measurements, one or more sites can be engineered with a known reference molecule. The reference molecule can provide an internal force calibration during the measurement.

### 1.7.4 Library of Sequences

One method for moving from force data to interpretation is to compare data from a sample molecule to a library of known sequences. In one embodiment, the library of known sequences is created using block copolymers. In one embodiment, the blocks are strings of the same nucleic acid. In one embodiment, the blocks contain more than one nucleic acid. The blocks may also be made of proteins, other biological macromolecules, synthetic polymers, or some combination thereof. In one embodiment, nucleic acids are used that are “double palindromes.” That is, they are palindromic in the sense typically referred to in nucleic acids, where the sequence reads the same from the 3′ as from the 5′ end, and they are also palindromic in the sense that a written word or sentence is a palindrome. That is, a single strand reads the same backwards and forwards. In fact, these double palindromes are not quite palindromes, but are functionally palindromes because of the repetitive nature of the DNA. For instance:

3′ . . . TGGCCACCGGTGGCCA . . . 5′
5′ . . . ACCGGTGGCCACCGGT . . . 3′

There is a sequence on the 3′ to 5′ strand that appears in the 5′ to 3′ strand, but offset.

Another strategy for learning sequences is to embed a known sequence in a long homopolymer. For example, one such sequence may be 3′ . . . CCCCCCGATCCCCC . . . 5′ Then, as it is read, the signal from the homopolymer section will be homogeneous, and the signature from the region that is different (in the example, GAT) will be distinguishable and can be extracted.

The library may include known signatures from each individual base or monomer, a library of all the known pairs of two, or three, or more. The library may be used to train a supervised learning model, into which the measurements on unknown sequences are fed. In one embodiment, force records from several known sequences are combined in some way to produce a stereotypical curve that may be compared to force curves from unknown sequences. For example, the curves from known sequences may be decomposed into principal components. In one embodiment, parameters are extracted from the force curves using known sequences. These parameters may include force value; first, second, or higher order derivatives; plateau length and height; parameters for fitting to a polynomial; power in a given frequency, for instance by taking a Fourier transform; or other features of a curve. In one embodiment, these parameters are then compared to the corresponding parameters from the force curve on the unknown sequence.

1.7.5 Identifying Bases from Data

Identification of a base or a number of bases in one sequence can be achieved by comparison of the force profiles from two sequences. In one embodiment, a force curve from an unknown sequence is compared to a library derived from force curves on known molecules. Supervised machine learning models can be trained using force curves on known sequences, then the model used to assign the sequence. A number of algorithms exist that can be used to compare the test waveform to the library, for instance those that have emerged from the field of speech recognition. Types of supervised learning algorithms that may be used include support vector machines, Markov models, random forests, Naïve Bayes algorithms, and neural networks. Unsupervised, semi-supervised, or other types of models may also be used. Model informed machine learning uses known physical characteristics of the system to create an algorithm. In one embodiment, model-informed machine learning is used to develop a base calling algorithm.

Machine learning and artificial intelligence may be used to determine how force profiles map onto data generated from the force-based sequencing experiment. These may be supervised, unsupervised, semi-supervised, or other classes of machine learning, such as reinforcement learning. In one embodiment, a reinforcement learning algorithm is used to teach the identification agent how to read the force profile. The force profile for a known sequence is measured. Then, the agent generates a predicted force profile, using one or more of the parameters described above, or other metrics. This predicted force profile is compared to the measured one, using an algorithm that measures similarity, for instance, dynamic time warping. The agent is rewarded more for predictions that are nearer to the observed force profile, and adjusts its predictions to increase its reward. Ultimately, the agent converges on a method for mapping the force profile onto the sequence. The convergence may take data from many force profiles on many different sequences. This mapping may then be used to identify bases in unknown samples. A genetic algorithm may be similarly employed. More than one learning technique may be used to generate the model.

In one embodiment, force recordings are broken down into sections that consist of small numbers (1-100) of bases. In one embodiment, these sections are sequential; alternatively, they may overlap one another. In one embodiment, rather than using raw data, each section is parameterized. These parameters may include force value; first, second, or higher order derivatives; plateau length and height; parameters for fitting to a polynomial; power in a given frequency, for instance by taking a Fourier transform; or other features of a curve. In another embodiment, no a priori parameterization is used, but rather the raw force recording section itself is used. Combinations of these approaches may also be used. A number of similarity measures exist to compare time series data. In one embodiment, a similarity measure is used to compare force records from a molecule under study to force signatures gathered from known sequences. For instance, dynamic time warping is a common algorithm for measuring the distance between two time series curves. In another instance a cross-correlation between the two time series curves is computed. Once the similarity between each pair of force recording sections has been calculated, the sections can be clustered. Each cluster will correspond to one base or short sequence of bases. This scoring and clustering can be done with available software, for instance the Tsclust package in the R programming language. The clusters can then be further compared to the library of known sequences in order to assign each cluster to a sequence.

1.7.6 Recognizing Rather than Sequencing

In some embodiments, the goal is to recognize a molecule or molecular structure rather than a sequence, or to recognize the presence or absence of other molecules. In this case, many copies of each known molecule, rather than each sequence, are translocated across the interface using the same conditions as the test. The force recordings from the known molecule are then treated as described for the known sequences above, and compared to an unknown molecule as described above. This classifier strategy is common in many areas of machine learning. For instance, it is a similar approach to that used in facial recognition.

### 1.7.7 Sample Preparation for Force-Based Sequencing

Force-based sequencing is compatible with a wide range of sample preparation methods, including methods that have few or no steps prior to the sample being introduced into the instrument for sequencing. Starting with a blood sample, tissue biopsy, cell pellet, cell scraping, or other sample taken directly from a human, animal, cell culture or plant, the sample is treated with a denaturing solution under conditions that lyse or break open the cells or tissues in the sample. The sample may also be treated with a non-denaturing solution. The denaturing solution also denatures any double stranded nucleic acid into single strands. This sample is then filtered through a membrane with pores with diameters <1000 nm, <500 nm, <200 nm, <100 nm, <50 nm, <10 nm. Or the sample is centrifuged to remove cellular and other debris from the sample. For sequencing of genomic DNA this sample may be sheared to a suitable size (0.5-2 kb, 1-5 kb, 2-10 kb, 5-20 kb, 10-100 kb or 20-500 kb) or the sample may be used unsheared. For sequencing rRNA or mRNA the filtered/centrifuged sample can be used directly without shearing. This sample preparation can be automated so that >100, >1000, >10,000, >100,000 samples can be prepared in parallel, in series or in combination. These samples are introduced into one or more of the fluids in which the analysis is performed.

Samples that can be sequenced by force-based sequencing include aptamers, aptamers with modified bases and SOMAmers. These molecules can be sequenced in their entirety, or a portion of the molecule sufficient to identify it can be sequenced. These molecules can be sequenced for the purpose of counting the relative or absolute number of aptamers, aptamers with modified bases or Somamers in a sample.

Samples for force-based sequencing can also be purified prior to sequencing. Purification can include extraction with phenol to remove proteins and ethanol precipitation. Purification can also include silica or glass-based columns or magnetic beads (e.g. AMPure XP).

Samples are purified or not purified can further be prepared for sequencing by repairing the ends of sheared molecules and tailing them with dATP or another base. When a specific end chemistry is desired an end modified oligonucleotide can be ligated to the sample molecule. The oligonucleotide modification can include a biotin, azide, NHS ester, alkyne, digoxigenin, amino group, thiol, dithiol or other known moiety or functionality.

Samples can be amplified prior to sequencing using the polymerase chain reaction, rolling circle amplification, loop mediated isothermal amplification, or other amplification method. Molecules are first sheared into a suitable size (0.5-2 kb, 1-5 kb, 2-10 kb, 5-20 kb, 10-100 kb or 20-500 kb), the ends repaired and tailed, followed by ligation of amplification primers to the ends of the molecules. For rolling circle amplification the sample molecules may be ligated to form a circular template prior to amplification. Primers for amplification or handle addition can also be inserted by transposon mediated chemistry. Samples can also be fragmented using transposon mediated chemistry.

Samples to be sequenced can be subjected to sequence expansion. Sequence expansion is a process by which each base in a sample is “expanded” such that there are two or more of the same base in a row, or bases in the original sample are replaced with a polymer or other structure that has a property that allows the identity of the original base at a given position to be determined. Such expanded sequences are sometimes referred to as Xpandomers. Expansion of a sequence relaxes the resolution requirements from single bases to the expansion size, which is less than or equal to 2 bases, 4 bases, 8 bases, 16 bases, 32 bases, 64 bases, 128 bases, 256 bases, 512 bases, 1 kbase, 10 kbases, 100 kbases, or the distance 1 nm, 10 nm, 100 nm, 1 μm, 10 μm, 100 μm. For force-based sequencing the expanded region or Xpandomer may be chose as to increase or optimize the force signature and/or base identification.

### 1.8 Indirect Sequencing

Direct versus indirect sequencing methods are distinguished based on whether or not the sequence of a nucleic acid is determined directly from the sample molecule or indirectly from some event or events that depend(s) on the sample molecule. Thus, Sanger sequencing and all methods that employ a polymerase or other enzyme to synthesize (or assemble) a molecule using the sample as a template, and use the synthesized molecule or information from the synthesis to determine the sequence of the sample molecule, are indirect methods. Nanopore sequencing, on the other hand, can be direct, since sequence is determined from the signal produced by the bases in the sample molecule as they pass through a pore. Force based sequencing is a direct sequencing method where the sequence of a molecule is determined by the base specific force profiles produced when the molecule is translocated through an interface from one chamber to one or more additional chambers. But force-based sequencing can also be implemented as an indirect sequencing method. For example, a single stranded nucleic acid sample to be sequenced is attached to the tip in the lower chamber. This molecule is then primed with an oligonucleotide that hybridizes to the sample, and the molecule is translocated across an interface until a force-based signal is detected from the oligonucleotide. The molecule is then returned to the first chamber, and a polymerase together with one of the dNTPs is introduced. If the dNTP introduced does not match the next base after the oligonucleotide primer, a subsequent force profile will look the same as the first one. If the base matches the sequence it will be incorporated by the polymerase and the force profile in the next translocation across the interface will reflect that incorporation and allow the relevant base to be assigned a position in the sequence of the sample molecule. Sequencing can also be performed on a fully double stranded DNA molecule attached to a tip. In the first chamber the DNA can be enzymatically or chemically nicked, producing a break in the DNA with a free 5′ end and an adjacent 3′ end. The density of the nicks can be controlled by controlling the nicking conditions. The molecule is then translocated across an interface until the presence of a nick is detected by a change in the force profile. The molecule is then returned to the first chamber, and DNA polymerase I or other polymerases that have a combination of polymerase and exonuclease activity can be introduced together with one of the dNTPs is added. The molecule is translocated again. If the base is successfully incorporated this will be reflected in the force profile and the base at the position in the sample molecule can be assigned. If the base is not successfully incorporated the profile will also reflect that, and the polymerization step is repeated with a different base. Enzymes that have sufficient displacement activity such the Klenow fragment of DNA polymerase I and the Phi29 polymerase can also be used in lieu of an enzyme that digests the DNA in advance of the polymerization, as they will displace the DNA in advance of the polymerization. However, an enzyme with exonuclease activity has the advantage of not producing a growing displaced single strand that will affect the force profile. This approach can also be used to detect ligation events, thus enable force-based sequencing by ligation.

### 1.9 Template for Development, Testing, Validation and Calibration of Sequencing Technology

The development of a nucleic acid sequencing method requires nucleic acid molecules for testing, validation and calibration of the new method. A sequencing method requires two things the ability to identify a base and the ability to resolve base identifications. For development purposes it can be desirable to separate these two requirements, allowing for example base identification to be developed without the simultaneous constraint of single base resolution. To fully separate the question of being able to identify a base from being able to resolve two adjacent bases from each other, test molecules composed entirely of a single base can be used. For example, a single stranded DNA molecule composed solely of dA, dT, dC or dG of a length from 2 bases to 200,000 bases can be used to establish the sequencing signal associated with the respective base. Molecules can also be made with mixtures of two or more bases in a way that allows for development of base identification methods and capabilities of a technology, while greatly relaxing resolution demands. For example, a molecule that has repeating blocks of 100 dA's and 100 dT's (either double stranded or single stranded) can be used as a development and test molecule. This block molecule provides a test for whether or not dA can be distinguished from dT while requiring only a resolution of 100 bases. Other combinations of bases such as dC and dG, dT and dC, dT and dG, dA and dC and dA and dG can be used to test for the ability to distinguish other bases from each other. These block nucleic acid molecules can also be composed of 3 or more different bases, in different orders. Once base identification has been achieved using a 100 base block molecule the resolution demand can be gradually increased by shortening the lengths of the blocks, so that 100 base long blocks could be reduced to 50, then 25, then 10, then 5 then 2 and finally 1. The gradual or small incremental decrease in block size allows resolution related technical and methodological issues to be addressed as they emerge without demanding single base resolution. Then advancing the resolution to 1 base and the ability to sequence a nucleic acid. Note that the single base resolution is only required for the case where identification is for one base at a time. Some sequencing methods involve reading out more than one base at a time, and those methods need not require single base resolution. For example, if a sequencing method can distinguish the 16 possible dimers of the four conventional DNA bases (dAdA, dAdC, dAdT, dAdG, dCdA, dCdC, dCdT, dCdG, dTdA, dTdC, dTdT, dTdG, dGdA, dGdC, dGdT and dGdG), then a resolution of two bases can suffice. Likewise, if a method can distinguish between the 64 different possible trinucleotides a resolution of three bases can suffice. A resolution better than what is required allows for a level of redundancy in the sequence determination, depending on how much better the resolution is than what is required. For example, single base resolution of a measurement when trimers are distinguished allows each base in a nucleic acid chain to contribute to three overlapping trimers.

Long block DNA molecules may be produced using synthetic circular double stranded DNA molecules from synthetic or natural oligonucleotides that serve as templates for rolling circle amplification. The circular template for diblock molecule that is composed of 50 base pair long blocks of dA:dT and dT:dA can be made from an oligonucleotide composed of (dA:dT)₂₅(dT:dA)₂₅ with 1-6 base cohesive ends made by adding bases to on one side of one strand and removing an equal number of bases (in the synthesis) from the opposite side of the other strand. This oligonucleotide is then ligated with a molecule that is 25 dT:A and 25 dA:dT, with cohesive ends corresponding to the first oligonucleotide. Exhaustive ligation of these two oligonucleotides produces a circular DNA molecule composed of 50 base pair long blocks of dA:dT and dT:dA. Blocks can be made longer or shorter, including 100 bases, 25 bases, 10 bases, 5 bases, 2 bases and 1 base long. The oligonucleotides can also be made with a smaller or no overlap between two adjacent blocks. To facilitate proper annealing of the block DNA oligonucleotides and ligation of these molecules into a circular molecule a short (1-20 bases) border can be added. This locked border also expands the number of available restriction sites that can be engineered in to the molecule for analytical or other purposes, as well as presenting unique priming sites for RCA and other amplification methods.

Long block nucleic acid molecules can be produced from the circular block template by rolling circle amplification (RCA). By using primers with modifications may facilitate the attachment to the manipulator, as described above. Single base identification can be performed by seeding one or a small number of bases into a known or homogenous block background.

Block DNA molecules can also be produced from signal stranded or RNA. A single stranded DNA molecule that is dA₅₀dT₅₀dA₅₀dT₅₀ (or any other combination of 2, three or four different bases) can be chemically synthesized. T4 RNA ligase or another ligase that can be used to ligate single stranded DNA or RNA is then used to ligate the ends of this molecule to form a circle, or to form a dimer, trimer or higher multimers. The dimers and other multimers will then be further ligated until they also form circular molecules. A short (10-50 bases) oligonucleotide that bridges the two blocks at the ligation junction can be used to facilitate more efficient ligation. These single stranded circular templates can then be used for RCA to produce a long (>the length of one circle) single stranded product complementary to the base sequence in the template.

A nucleic acid molecule with a periodic sequence that when subjected to an analysis produces a signal with the same periodicity demonstrates that the analysis is base dependent. The periodic structure of these block molecules also facilitates the detection of weak base dependent signals by allowing averaging methods to be used to improve the signal to noise.

### 1.10 Protein Sequencing

Force based analysis can be used for determining the sequence of amino acids in proteins or polypeptides, as well as modifications to amino acids in a polypeptide, using techniques described for DNA elsewhere in this document. A polypeptide chain translocated through an interface will produce a force profile that depends on the composition and order of amino acids in the polypeptide, as well as any structure adopted by the polypeptide chain. Protein sequencing can be performed by attaching either the N-terminal end or the C-terminal end of a polypeptide to a tip via, for example, either amine or carboxy specific chemistries, such as NHS chemistry for the N-terminus or carbodiimide chemistry for the C-terminus, which will produce some end attached molecules. Depending on which part of the molecule is attached to the tip, a unique force profile will result when the molecule is translocated through the interface. This profile may then be stored, viewed, and analyzed as described elsewhere in this document. Under conditions where the molecules are attached by one end or the other and denatured, the protein is then translocated through an interface and the force profile recorded. The force profile is then analyzed to determine the sequence of the protein or the presence of modifications to the protein. Alternatively, a protein can be attached by either the N-terminal end or the C-terminal end to another polymer, such as a nucleic acid, polyethylene glycol, a polysaccharide or similar polymer, with the polymer attached at the other end to the tip and acting as a spacer between surface of the tip and the end of the protein molecule. The polymer-protein is then translocated through an interface with the tip until the protein reaches the interface, at which point the force profile recorded will reflect the type and order of amino acids in the protein as they move through the interface. This a direct single molecule sequencing that can be used to establish the order of amino acids in one or more unknown proteins, as well as determine the protein composition of a sample. Force based protein sequencing can also be used to directly determine which splice variants of a protein that are expressed, as well as which amino acids on an individual protein have modifications such as phosphorylation, myristylation, ADP-ribosylation, ubiquitination, actetylation, farnesylation, disulphide bond formation, sulfation, gamma-carboxylation, glycosylation (monosaccharides as well as oligosaccharides), proline isomerization, hydroxylation, sumoylation and others. Force based protein analysis can be used for research and development purposes, including the development and validation of therapeutic molecules (including but not limited to therapeutic proteins or peptides). Force based protein analysis can also be used for quality control purposes for proteins used to medical, industrial or research purposes. For example, the force profile associated with the translocation of a protein across an interface will depend on whether or not the protein is properly folded. Thus, force-based analysis can be used to quantify the extent of denaturation in a protein sample.

### 1.11 Other Applications

The polymer to be sequenced may be one of the following: DNA, RNA, protein nucleic acids, proteins, polysaccharides, xeno nucleic acids, block copolymers, or other synthetic polymers. They may also be a combination of the above, for instance a glycosylated protein. The polymer may contain regions that are ordered/structured and regions that are unstructured. The technique may be used to sequence biological macromolecules originating from viruses or any living thing, or synthetic molecules. It may be used in a clinical setting, by a person for their own personal use, or in a research setting.

Larger structures made of macromolecule subunits may be studied using the technique, including protein structures such as actin, intermediate filaments, microtubules, amyloid fibrils, and virus capsids.

The technique may be used to measure quality of synthesized polymers or fibers, where a polymer or fiber is pulled through an interface after manufacture. Since contact angle depends strongly on the properties of the material, changes in the contact angle could signal changes in the manufacturing process.

In a single-cell assay, entire bacteria can be pulled through the interface.

A plate such as those used for ELISA assays can be pulled through the interface. In this application, antigens, aptamers or Somamers that bind a specific protein are attached to a surface in a known pattern. When a protein binds, the surface becomes rougher, changing the force required to pull the plate through the interface.

Langmuir-Blodgett troughs are used to create thin films for a variety of applications. In this technique, a film of the desired material is introduced at a fluid-fluid interface. This interface is typically aqueous-air, though other combinations of fluid are sometimes used. The film is formed and its properties can be controlled by moving one or more ends of the trough to change its surface area. This change in surface area in turn controls the molecular packing of the film. The film is then transferred to a surface, or alternatively studied in situ. A Wilhelmy plate is a calibrated plate that is dipped into a Langmuir-Blodgett trough to measure the surface or interfacial tension σ through the measurement of the force on the plate F and the contact angle Θ. Specifically, σ=F/(I*cos(Θ)), where I is the cross-sectional perimeter of the plate. When this is done during synthesis of the Langmuir-Blodgett film, it can be used as a quality control mechanism. In one application of the present invention, a molecule or group of molecules suspended through an interface is used as a nanoscale Wilhelmy plate to measure the interfacial tension of the fluid-fluid interface, or to monitor it during adjustment of the interface. Force is measured as described elsewhere in this patent, and contact angle is measured by optical means. For example, the contact angle with the molecule can be measured by the “optical lever” method, where a beam of light is introduced in such a manner that it scatters from the interface. The angle of the scattering is dependent upon the contact angle, and the ultimate position of a spot at the other end of the beam some distance away is, in turn, dependent upon the scattering angle. The spot position can be measured by collecting the light on a position-sensitive detector. The adjustment of the interface could be due to addition or removal of molecules, or to their reconfiguration, for instance by changing lateral pressure as in a Langmuir-Blodgett film. This measurement can be done with force sensitivity less than 1 pN, less than 10 pN, less than 100 pN, less than 1 nN, less than 10 nN. The measurement can be used to inform a feedback loop that dynamically adjusts the interfacial tension. Molecules may be engineered, or natural molecules selected, for sensitivity in the correct range of interfacial tension.

The roughness of a surface can be quantified by translocating it through an interface, and comparing the force signature to that of the same surface before or after polishing, by comparing the force signature to force signatures of surfaces with known roughness, by comparing the force profile with one predicted from theory or computation.

### 1.12 Non-Sequencing Applications

### 1.12.1 Small Molecule Binding to Nucleic Acids

Many small molecules associate with nucleic acids through predominantly non-ionic interactions or mixed ionic and non-ionic interactions, and such molecules will also produce a detectable change in the force profile when the nucleic acid is translocated through an interface (compared with the force profile when the small molecules are not there). These include molecules such as the intercalating dyes ethidium bromide, propidium iodide, and YOYO-1. These particular examples act as stains for nucleic acid by virtue of the fact that intercalation between two bases produces a large increase in fluorescence intensity. Many of these molecules have a preference for double stranded DNA, but will also associate with or bind to single stranded nucleic acids. TOTO-1 is a DNA dye that interacts with ssDNA. SYBR gold is another dye that is typically used to stain single stranded DNA in denaturing conditions, for instance gels containing urea and formaldehyde. SYBR green II is another dye that is particularly useful for RNA and can also bind to single-stranded DNA. The association or binding of intercalating dyes to single stranded nucleic acids is typically weaker than with double stranded molecules, although some molecules such as TOTO-1 are reported to have affinities for single stranded nucleic acids similar to those for double stranded nucleic acids. Intercalating dyes have varying degrees of sequence specific, ranging from almost none to dyes such as ACMA (9-amino-6-chloro-2-methoxyacridine) which has a high preference to d(AT) stretches of DNA. Thus, certain intercalators can be used alone or in combinations to identify specific nucleic acid sequence. Certain drugs are known to bind nucleic acids. For instance, quinolones are a class of antibiotics that have been shown to bind single stranded oligonucleotides in a sequence-dependent manner. Organic small molecules can be discovered to bind particular sequences, optimized for the task of binding specific ssDNA sequences, building upon what is known about existing single stranded binding small molecules. Other nucleic acids bind biological macromolecules, including DNA and RNA. These could also be nucleic acids with non-natural backbones (XNAs) or modified bases. Aptamers or Somamers can be discovered using the SELEX process to bind nearly any target, including sequences of DNA. Certain proteins and peptides bind single-stranded DNA. A number of single-stranded binding proteins exist in nature. In one embodiment, small molecules are bound to the molecule under study. These may remain bound through the course of the experiment; they may be removed during the experiment, for instance upon translocation through the interface; or some combination of these two. The small molecules may be dyes, drugs, or other molecules. They may include the molecules described above, or other molecules.

Molecules that bind in one of the grooves of DNA often employ a mixture of interactions, such as involving both ionic or electrostatic interactions in combination with hydrophobic or van der Waals interactions in combination with hydrogen bonding. Such molecules include the minor groove binding bisbenzimide dyes such as Hoechst 33258 and the antibiotic distamycin-A, as well as major groove binding molecules such as methylene green. In one embodiment, ions or molecules bound to the molecule under study interact specifically with molecules confined to the interface. For example, a molecule bound to DNA might form a stable structure with molecules at the interface, which is then broken as the molecule is moved through the interface, generating a change in force.

### 1.12.2 Screening for Nucleic Acid Binding Molecules

Nucleic acids are important targets for drug development, and a central aspect of identifying drug candidates for these types of targets is establishing that a molecule binds to a nucleic acid, modifies a nucleic acid, facilitates the binding of one or more other molecules or facilitates the modification of a nucleic acid. Because any molecular binding to a nucleic acid or modification of a nucleic acid will alter the force recording of a nucleic acid relative to the force recording without the binding/modification, a force-based assay can be used to screen for molecules that bind directly or indirectly to nucleic acids and may modify the structure or function of the nucleic acid. Such screening can serve to identify molecules or compounds that have therapeutic uses or that provide lead compounds for the development of therapeutic molecules or compounds. The force-based screening for nucleic acid binding/modifying molecules can be performed by flossing a target DNA molecule and during or between flosses introduce the molecule or group of molecules to be tested. Changes to the force profile between before the molecule or molecules are introduced and after are measured to determine if the DNA has been modified. The sample compartment can then be rinsed to remove the molecule or molecules introduced, and the process can be repeated with a new molecule or collection of molecules. Alternatively, molecules can be introduced in series to determine the order of binding/modification of more than one molecule to the nucleic acid.

The screening can be performed in a parallel fashion using an instrument that has two or more measurement systems. The parallel approach can be used to expand the throughput of the serial approach described above, or it can be used to make parallel measurements for comparison. For example, one measurement system can have no molecule or molecules to be screened while a parallel measurement is performed with the molecule or molecules to be tested present. Measurements from these two systems are then compared to determine if binding or modification occurred.

The target nucleic acid molecule used above can be selected or designed for the specific question being addressed. A search for a non-specific nucleic acid binding molecule can be performed with a random or non-specific DNA molecule such as sheared salmon sperm or calf thymus DNA, or with smaller DNA molecules with defined lengths such as linearized pBR322 or linear lambda phage DNA. Highly directed searches for a molecule or molecules that bind to one or more specific sequences can be performed be engineering the sequence of interest into a non-specific background. This segment can be repeated at some predefined distance such that binding/modification would produce a periodic or non-periodic signal in the force profile. In addition, a fiducial force signature can be engineered into the test molecule to facilitate identification of binding/modification of a specific sequence. The nucleic acid can also be designed to screen for specific binders to an unknown or partially known sequence. Because binding/modification of DNA typically involves a relatively short segment of DNA (1-10 bases), a screening molecule can be composed of for 10, 100, 1000, 10,000 or 100,000 unique 10 mer sequences concatenated into one DNA molecule. When the DNA molecule is translocated across the interface the location of any changes in the force recording relative a fiducial force marker would identify the binding/modification sequence.

Screening for nucleic acid binding molecules in many instances be accomplished without single base resolution in the force recording. In these cases, the resolution required for screening must be less than 2 bases, 4 bases, 8 bases, 16 bases, 32 bases, 64 bases or 128 bases. This relaxation can be achieved by placing the target sequences sufficiently far apart that they can still be resolved. Alternatively, the relaxation of the resolution requirement introduces some uncertainty as to which sequence was bound. The molecule or molecules can then be rescreened against sequences within the range of possibilities defined by the position of a signal and the positional resolution of the measurement. A relaxation in resolution required for some applications also relaxes technical specifications for the force-based detection instrument allowing for a less expensive instrument.

### 1.12.3 Protein Bar Coding

Proteins bound to double or single stranded nucleic acids will produce a force signature when the nucleic acid is translocated through an interface that is different than the force signature produced when there is no protein associated with molecule. This force signature will depend on the structure and sequence of the protein, how it is bound to the DNA and the composition of the medium on both sides of the interface. There are a wide range of well-known naturally occurring DNA binding proteins whose binding could be detected in this fashion. This includes type II restriction enzymes which in the absence of a suitable divalent cation will bind to their cognate sites but not cut the nucleic acid. Using restriction enzymes as binding molecules permits mapping of restriction sites on a nucleic acid from the force profile, or identifying fiducial marks or points of reference on a molecule. Further, zinc finger proteins can be designed and engineered to bind to almost any DNA sequence that fits within the zinc finger proteins binding site. By making modifications to the zinc finger protein away from the binding site, these proteins can also be designed to produce distinguishable force signatures at each binding position. Placing such zinc finger protein binding sites along a DNA molecule produces a two-dimensional bar code of position versus signal type/strength.

### 1.12.4 Immunoassay and Affinity Assays

Force based analysis can be used for immunoassays or related affinity-based assays. A polymer such as a nucleic acid, a polysaccharide, polyethylene glycol or other is used as a scaffold onto which an affinity reagent, such as an antibody (or some fragment thereof), an affibody or aptamer (including aptamers with modified bases and SOMAmers). The polymer is end attached to a cantilever, and the polymer chain with attached affinity reagents is submerged into control solution that does not have any specific ligands. The polymer is then translocated across the interface and a control force profile is established. The polymer is then moved back into solution and a sample to be tested is introduced. If there are ligands with specificity to the affinity reagents on the polymer, these will bind to some fraction of the affinity reagents. The fraction of affinity reagents with a bound ligand will depend the incubation time and the equilibrium constant of the ligand-affinity reagent interaction under the conditions of the assay. The polymer is then translocated across an interface. The force profile from this translocation will depend on the fraction of affinity reagents with a ligand bound and where those ligands are bound. This assay is repeated at more than 2 but less than 1000 dilutions to ensure that one or more translocations are performed when more than 0.1% but less than 99.9% of the affinity sites are occupied. The translocation data can be analyzed immediately after each dilution test to determine if the result is in a desirable range of the assay (0.1-99.9% as above, or any other user established predetermined range). If the measurement is in the range, the assay of that sample can be ended. Alternatively, the sample can at that point be returned to a solution without the ligand. After waiting 1, 10, 100, 1000, 10000 or 100000 seconds, the polymer can be translocated again. The reduction in the number of ligands bound can then be used to calculate or estimate the off rate of the interaction with the affinity reagent and the ligand.

Instead of affinity reagents, one or more molecule for which a binding partner is sought can be attached to a polymer. Translocation of the polymer from a control solution produces a reference force profile. One or more molecules for which binding to the target is to be assayed is introduced into the first medium. If any molecules in the medium bind to any molecules on the polymer, this will produce a change in the force profile. For example, a therapeutic target (protein) can be attached to the polymer and a small molecule from a natural or synthetic source can be introduced to the first medium (after the control translocation). If the molecule has affinity for the therapeutic target it will bind some fraction of the targets, which will be reflected in the force profile. The strength of binding can be evaluated by the concentration of the small molecules and the fraction of targets occupied. By using a large number of cantilevers in parallel or by testing molecules in series or by using a combination of cantilevers in series and serial testing, a large number of molecules (often referred to as a compound library) can be screened for target binding molecules.

In one implementation a single type of purified molecule is attached to the polymer and a single type of molecule to be tested is assayed as above. However, to increase throughput a mixture of 2-1000 different targets can be attached to a polymer and these can be used to test a solution that contains 1-10000 different binders. A positive signal would then lead to the targets and binders to be divided into smaller groups to be tested. The group that tests positive is then further divided until a single target-binder pair is identified. In some cases, there may also be cooperative binding that requires more than one binder to form a stable association with the target. Such binders would also be identified by the force assay.

In another implementation one or more affinity reagents are attached to a plate, bead or other solid object. The forces required to translocate the object across an interface are altered by binding of the affinity reagent to molecules or molecules attached to an object or attached to one or more additional molecules.

### 1.12.5 Miniaturized Evaporimeter

The measurement of rates of evaporation through evaporimetry is important in many industrial and commercial applications. Hydrologists for example rely on evaporimetry when computing water flux, and evaporimetry is important for maintaining specific levels of humidity is many research and production environments. A polymer or small object attached to a tip, where force on the tip is monitored while the polymer or object is translocated across the interface provides for a miniaturized evaporimeter. The movement of the interface due to evaporation or condensation will cause the force profile to shift in a measurable fashion, thereby allowing the rate of evaporation or condensation near the polymer or object to be determined. Evaporimetry using this approach can be performed locally, as well as in small chambers or cavities that are fabricated or form naturally in various materials. The size of the openings in the cavities or chambers that can be examine can have a smallest dimension of <1 cm, <0.1 cm, <0.01 cm, <1 μm, <0.1 μm, <0.01 μm, <1 nm.

### 1.12.6 Solvent Quality Testing

A polymer immersed in a solvent has properties that depend on the balance of interactions of the polymer with itself, the polymer with the solvent and the solvent with itself. Polymers in solvents where attractive interactions between different polymer molecules or between different parts of one polymer molecule are close to equal to polymer-solvent attractions will form a random coil, where the shape of the coil is determined primarily by the stiffness of the molecule and thermal energy. Polymer-polymer interactions and polymer-solvent interactions do not play a significant role. This is called the theta condition for a polymer and solvent combination. If the polymer-polymer attractive interactions are large relative to the polymer-solvent interactions the random coil formed by the polymer will tend to collapse, leading to a smaller radius of gyration. At the point where such attractive interactions overcome the motions induced by thermal energy the polymer will collapse such that different parts of the molecule are in sustained contact with other parts of the molecule or other polymer molecules to form an aggregate. If on the other hand the solvent-solvent attractive interactions are large relative to the polymer-solvent interactions (the equivalent of polymer-polymer interactions being repulsive relative to each other) the random coil formed by the polymer will expand to a radius of gyration larger than that for the theta condition. One continuous measure of the polymer-solvent interaction is the Flory parameter X (chi). The solvent quality for a polymer is an important parameter used to characterize polymer-solvent combinations for industrial, pharmaceutical, commercial and research purposes. The force-based assay can be used to characterize the solvent quality for a particular polymer by measuring the force required to translocate the polymer to be tested from one solvent to another or from one solvent into another medium. The solvent quality will affect the structure and conformation of a polymer, as well as the interaction that the polymer has with an interface. These effects will be reflected in the force profile. The force-based assay can be used to measure solvent quality one molecule at a time, thus providing more variance of solvent-polymer interactions that underpin the solvent-polymer interaction from bulk measurements such as light scattering. Further, the force-based assay can be used to measure how the solvent quality varies as a function of position on a polymer. The solvent quality models used generally treat a polymer as being homogeneous along its length. However, on a sufficiently small length scale all polymers are inhomogeneous and the solvent quality will vary locally along the length of the molecule. This local solvent quality heterogeneity reduces the accuracy and utility of models based on homogeneous polymers. Thus, these measurements can be used in more detailed models that better predict the behavior and properties of a polymer under different conditions.

Force records can also be used to measure polymers in a sample, for instance contamination by DNA. For example, with proper attachment technique, a sample free of polymeric material examined with the FBS technique yields a trace that shows recovery to zero force when the probe is still near the surface, rather than the long force plateaus observed in the presence of polymeric material. Single stranded and double stranded DNA each produce characteristic force records comprising long plateaus with a final step to zero force. The height of this step is dependent on the structure of the DNA, and the plateau length is indicative of the length of the contaminant. The force record characteristics for different polymers can be well-calibrated from known samples, so that a wide variety of information can be gained. The technique can also be used to test for the presence of other molecules. As described above, many different types of additives, including ions, surfactants, small binding molecules, or other polymers, bind to the DNA in a way that changes the force profile. Thus a known DNA can be used to measure their presence.

### 1.13 Oligonucleotides and Related Molecules

Oligonucleotides and related molecules such as protein nucleic acid (PNA) molecules can be designed to associate or bind to a complementary sequence. This association or binding can occur via Watson-Crick base pairing to form duplex molecules as well as Hoogsteen base pairing to form triplex or quadruplex nucleic acid structures. These associations of oligonucleotides with nucleic acid molecules produce changes to the force profile of a nucleic acid translocated across an interface. Oligonucleotides and related molecules can be used to produce a bar code signature. They can also be used for identification of specific sequences in a sample, for research of clinical (diagnostic) purposes. DNA and other nucleic acids can be assembled into complex structures using so called DNA origami, or nucleic acid orgami. The structure and organization of such complex structures can be examined by translocation across an interface and examining the force signature. Origami structures can also server as scaffolds for affinity reagents or other molecules in applications described above.

### 1.14 Force Labels and Contrast Agents

While there are many applications of force-based detection that are based on directly measurable forces, there are also instances when it is beneficial to enhance or alter the signal produced using additional molecules or structures attached to the nucleic acid or other molecule or structure being translocate. Such force-based labels/contrast agents are composed of a molecular, group of molecules, a particle or other object that attached to something being translocated in order to increase or change the signal produced in some way. The labels or contrast agents are specifically designed to produce forces or force profiles that are unique or distinguishable.

### 2 Exemplary Methods and Apparatus

Methods and apparatus according to the present teachings may include various techniques and equipment for determining one or more structural features of a molecule by measuring forces as the molecule is caused to cross a fluid interface. Some exemplary teachings are further described without limitation in this section.

A method of sequencing a polymer may include generating a curved interface of a first fluid containing the molecule to be sequenced in contact with a surrounding second fluid. An attachment is made to the polymer and a force is then applied to the polymer molecule sufficient to cause the molecule to be translocated across the curved interface, and the applied force is analyzed to determine an internal structural feature of the polymer molecule. In some case, this can include sequencing some or all portions of the molecule. Interfaces formed according to the present teachings can have any suitable radius of curvature, such as one millimeter or less.

The fluid within which the molecule to be sequenced is immersed may in some cases be termed a foreground fluid, whereas the fluid toward which the molecule moves as it crosses the fluid interface may be termed a background fluid. In some cases, these fluids may simply be termed first and second fluids. In some cases, both fluids may be liquids. In other cases, one or both fluids may be gases or vapors. In some cases, the molecule to be sequenced may be pulled from a liquid into the air, and the air may have a controlled degree of humidity.

The medium within which molecules to be sequenced may take the form of a spherical drop, but may take other shapes. In some cases, the molecule to be sequenced may be contained in a truncated, largely spherical drop held on a surface. In some cases, molecules to be sequenced may be contained in a reservoir of fluid bounded by either a curved interface or a substantially planar interface. Curved interfaces can be formed, for example, by forcing fluid through a narrow aperture, such as the tip of a needle or micropipette, or through a small aperture in an otherwise solid barrier, such as porous graphene or some other suitable barrier material, additional examples of which have been provided in the present disclosure. In some cases, the curvature of the fluid interface may be kept constant as the molecule to be sequenced is translocated across the interface, while in other cases, the radius of curvature may vary.

Molecules to be sequenced may be caused to cross a fluid interface in a variety of ways, such as with a controllable manipulator of some type. This may take the form of cantilever tips (similar to an atomic force microscope tip), optical tweezers, magnetic tweezers, or any other mechanism for manipulating molecules. In some cases, an array of such controllable manipulators may be provided, to manipulate a plurality of molecules simultaneously.

To discern properties of the molecule to be sequenced, a force sensor of some type measures the force applied to move the molecule across a fluid interface. The term “force sensor” refers to any method or apparatus for measuring this applied force or forces, typically as a function of the position of the molecule as it crosses the fluid interface. One possible class of force sensors are microfabricated transducers, such as cantilevers, which measure forces based on deflection of light from the tip or by piezoresistivity. However, any manner of measuring the forces applied to a molecule may be suitable.

In some cases, amphiphilic molecules such as surfactants may be disposed at the fluid interface. This can be the case at the boundary of a fluid droplet or other curved interface, or at a substantially planar fluid interface. Such amphiphilic molecules may be configured to interact with the polymer molecule(s) to be sequenced, to enhance the ability to determine properties of the polymer. For example, interactions with amphiphilic molecules at the fluid interface may help to differentiate between possible components in the polymer to be sequenced, or may increase the overall signal to noise ratio of the force measurements.

In some cases, additional molecules may be added to one or both fluids on either side of the fluid interface, for the purpose of altering the measuredforce profile as the molecule to be sequenced is translocated across the fluid interface. Such molecules may sometimes be termed “secondary molecules.” Many types of secondary molecules may be used, and in some cases the molecules may be positively or negatively charged ions. Many possible examples of secondary molecules are provided in this disclosure. In general, the interactions between secondary molecules and the molecule to be sequenced may help to distinguish components of the molecule to be sequenced.

In some cases, the force applied to a molecule to cause it to be translocated across a fluid interface may be modulated at one or more known frequencies. This can be accomplished in a variety of ways, such as by modulation of the primary manipulator, modulation of a separate actuator, or modulation of the interface itself. Modulation of this type coupled with a phase-sensitive amplifier may in some cases be termed “lock-in amplification.” This technique may introduce frequency components in the force that are related to the local derivative of the force versus position profile of the molecule to be sequenced. This signal can be detected using phase coherent detection such as that used in a lock-in amplifier. Such a signal provides a higher signal-to-noise ratio than the original force signal.

In some cases, a biological macromolecule may be denatured before sequencing, by providing a denaturing agent or method step. For example, DNA or RNA may be denatured through the application of heat, or by exposure to a wide variety of denaturing agents, examples of which are provided elsewhere in the present teachings. Denaturing may simplify subsequent sequencing of the separated polymer strands.

In some cases, molecules to be sequenced may have a proximal end attached to an object within the original fluid, for example, a magnetic trap. This causes the molecule to be straightened or held straight as it is translocated across a fluid interface, and can allow for more accurate linear sequencing as the distal end of the molecule is pulled through the interface. For example, the proximal end of the molecule to be sequenced may be attached to a magnetic bead exposed to a magnetic field tending to hold the bead within the first fluid.

In some cases, molecules to be sequenced, such as DNA molecules, may be pulled from a liquid into a gas such as air. This may help dehydrate the DNA and avoid or reduce having liquid wick up the strand. The more wicking there is, the more bases contribute to the force signature at any given time, which makes deconvoluting the sequence more difficult. Therefore, pulling a polymer into air may simplify the sequencing analysis.

### 2.1 Methods of Sequencing a Polymer

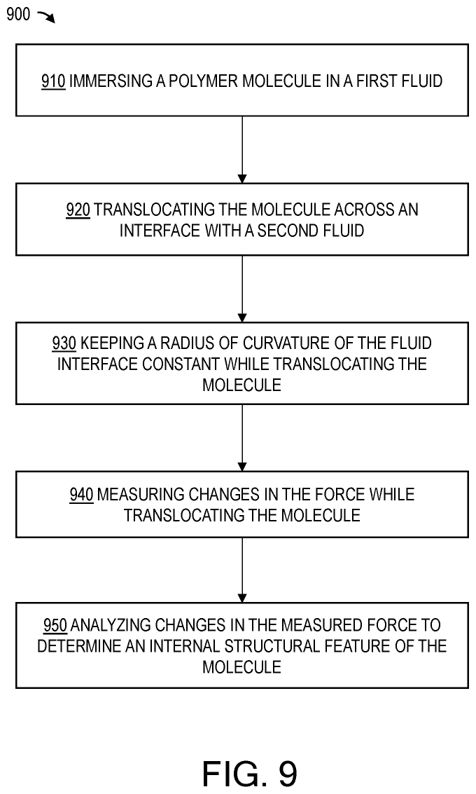

This section describes some specific exemplary methods of sequencing polymers according to aspects of the present teachings, with reference to FIGS. 9-11.

FIG. 9 depicts the steps of a method, generally indicated at 900, of sequencing a polymer. At step 910, method 900 includes immersing a polymer molecule in a first fluid. The polymer molecule and the fluid may be of any suitable type, including but not limited to any of those described in the present teachings.

At step 920, method 900 includes translocating the polymer molecule across an interface between the first fluid and a second fluid. As described previously, the translocation may be achieved by a manipulator, for instance by a magnetic bead or a cantilever tip to which the polymer molecule is attached. The second fluid may be a liquid, or in some cases it may be air or some other gaseous fluid or vapor.

The interface between the first and second fluids may have a known or predetermined radius of curvature. For example, the interface may have a radius of curvature of less than one millimeter in at least one direction, or less than one micrometer in at least one direction. The interface also may be formed in various ways. For example, the interface may be formed at a tip of a needle by pushing the first fluid through the needle. In some cases, the interface may be formed at an aperture formed in a barrier separating the first fluid from the second fluid. The barrier may, for instance, be formed of a polymeric material.

At step 930, method 900 includes keeping a radius of curvature of the interface constant while the polymer molecule is translocated across the interface. For example, as described previously, if one fluid is contacting another through an opening, the pressure on the fluid may be adjusted to create the desired radius of curvature. This pressure may be applied externally, or it may be a function of the interfacial tension, which is, in turn, determined by the chemical properties of the support structure, each fluid, and any molecules that are present at the surface. In the case of an aqueous fluid, the chemical properties determining the hydrophobicity and hydrophilicity of the support, barrier or walls of the opening may contribute to the curvature of the interface.

At step 940, method 900 includes measuring changes in the force while translocating the polymer molecule (e.g., changes in a force associated with the translocation of the molecule). This measurement can be accomplished using any of the various force sensors and sensing techniques described previously, or any other suitable measurement method. For example, a microfabricated AFM cantilever may be used, with forces measured based on piezoresistance of the cantilever. Examples of other suitable force measurement techniques include camera-based bead tracking, scattering-based bead tracking, measurement of local deflections at the fluid interface, using force-sensitive nucleic acid handles, detecting the movement of fluorophores at the fluid interface, and/or detecting pressure waves due to translocation of the polymer, all of which have been described in detail above.

At step 950, method 900 includes analyzing the changes in the measured force to determine an internal structural feature of the polymer molecule. For example, changes in the measured force may be used to sequence the molecular structure of the polymer as it is translocated across the fluid interface. For instance, in the case of a DNA molecule, different applied forces may correspond to the different nucleobases adenine, cytosine, guanine, and thymine, allowing sequencing of the molecule.

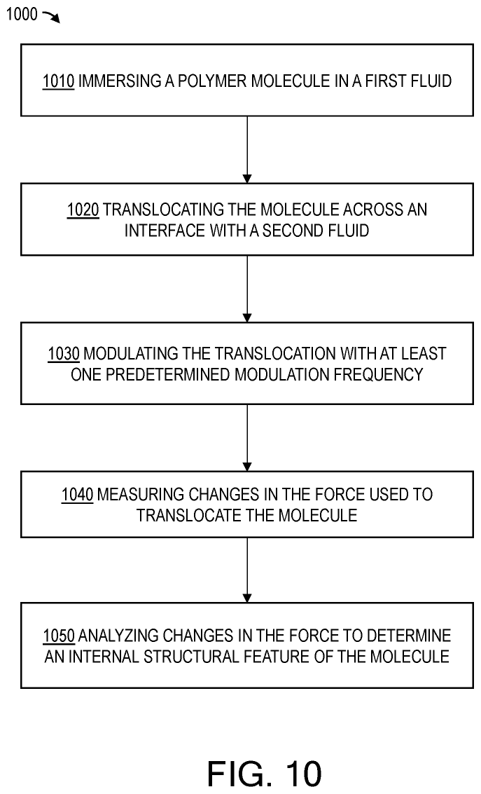

FIG. 10 depicts the steps of another method, generally indicated at 1000, of sequencing a polymer. Some steps of method 1000 may be similar to the steps of method 900 described above. At step 1010, method 1000 includes immersing a polymer molecule in a first fluid. The polymer molecule and the fluid may be of any suitable type, including but not limited to any of those described in the present teachings.

At step 1020, method 1000 includes translocating the polymer molecule across an interface between the first fluid and a second fluid. As described previously, the translocation may be achieved, for example, by a magnetic bead or a cantilever tip to which the polymer molecule is attached, and more generally by any movable device to which a polymer molecule may be attached. The second fluid may be a suitable liquid, or in some cases it may be air or some other fluid in gaseous form.

At step 1030, method 1000 includes modulating the translocation with at least one predetermined modulation frequency. For example, a small modulation of the translocation of the molecule, typically on the order of the size of the signal to be detected, may be introduced at a fixed frequency or multiple frequencies. The modulation can be produced by the same method which is used for translocating the molecule or by an additional actuator. This introduces a frequency component in the force, which can be detected at a significantly higher signal-to-noise ratio than the original force signal.

The frequency or frequencies of modulation can be chosen to be at a resonant frequency of the system, above a resonant frequency, or below a resonant frequency. The frequency or frequencies can be changed during the course of the experiment, or may be fixed. Signal amplification can be implemented in analog hardware, digitally in hardware, including on a reprogrammable hardware chip (e.g. FPGA), or using software.

At step 1040, method 1000 includes measuring the changes in force required to translocate the molecule across the interface, in a manner similar to step 940 of method 900, described previously.

At step 1050, method 1000 includes analyzing the changes in force to determine an internal structural feature of the polymer molecule, such as the molecular sequence of the molecule. Step 1050 may be similar to step 950 of method 900, described previously.

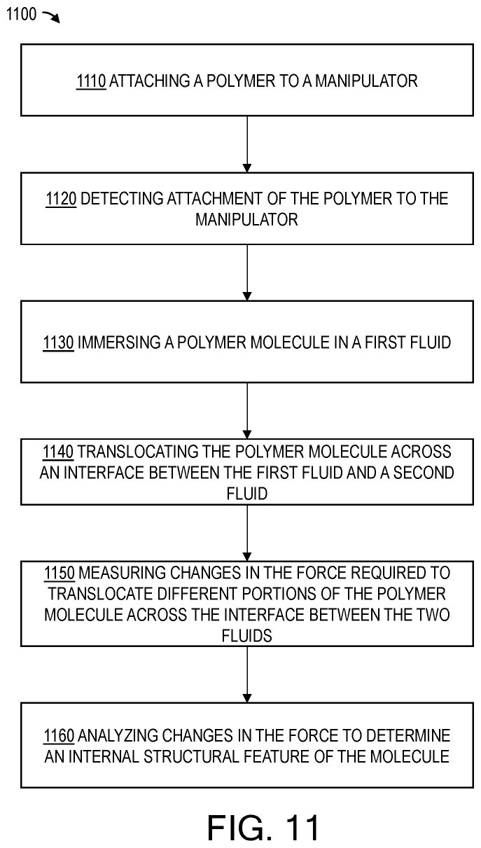

FIG. 11 depicts the steps of yet another method, generally indicated at 1100, of sequencing a polymer. At step 1110, method 1100 includes attaching a polymer to a manipulator. For example, the manipulator may be a magnetic bead, cantilever tip, other MEMS device, or some other movable device. The polymer may be attached to the manipulator, for example, by specific chemical attachment. Possible attachment mechanisms, as described previously, include various functional groups and specialized molecules that can be attached to the polymer molecules, the manipulator, or both. Examples include antibodies, aptamers, SOMAmers, functionalized oligonucleotides, and alkyne-containing molecules, among others.

At step 1120, method 1100 may include detecting attachment of the polymer to the manipulator. For example, during translocation across a fluid interface, an attached molecule exerts a force that can be distinguishable from a situation where no molecule is attached, several molecules are attached, or the manipulator is somehow fouled. Thus, detecting attachment of a single molecule may include, for instance, measuring a force when the manipulator is a desired height above the interface, and determining that the force is within a predetermined range corresponding to a single attached molecule. Similarly, detecting attachment of at least one molecule may include measuring a force when the manipulator is a desired height above the interface, and determining that the force is above a predetermined minimum.

At step 1130, method 1100 includes immersing a polymer molecule in a first fluid. The polymer molecule and the fluid may be of any suitable type, including but not limited to any of those described in the present teachings. In some cases, immersing the polymer molecule in the first fluid may be performed before detecting attachment of the polymer to the manipulator. In that case, detecting the attachment may involve translocating the molecule across an interface between the first fluid and another fluid. In other cases, detecting attachment may be performed previously, either by translocation or some other method, and the molecule may be immersed in the first fluid while already attached to the manipulator.

At step 1140, method 1100 includes translocating the polymer molecule across an interface between the first fluid and a second fluid. At step 1150, method 1100 includes measuring changes in the force required to translocate different portions of the polymer molecule across the interface between the two fluids. At step 1160, method 1100 includes analyzing the changes in the required force to determine an internal structural feature of the polymer molecule. Steps 1140, 1150 and 1160 are similar to the corresponding steps of methods 900 and 1000.

### 3 Illustrative Combinations and Additional Examples

This section describes additional aspects and features of display and image-capture devices, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.

### Curved Interfaces

A. A method of sequencing a polymer, comprising:

generating a curved interface between a first fluid containing a polymer molecule with a second surrounding fluid, the first fluid having a volume defined at least partly by the curved interface;

contacting and applying a force to the polymer molecule sufficient to cause the polymer molecule to be translocated across the curved interface;

measuring the applied force as the polymer molecule is translocated; and

analyzing the applied force to determine an internal structural feature of the polymer molecule;

wherein the curved interface has a radius of curvature of less than one millimeter in at least one direction.

B. A method of sequencing a polymer, comprising:

generating a plurality of droplets, each including a polymer molecule immersed in a surrounding liquid, each droplet having a volume defined at least partly by a curved outer surface;

applying a force to each polymer molecule sufficient to cause the polymer molecule to be translocated across the curved outer surface of the respective droplet;

measuring the applied force as each polymer molecule is translocated; and

analyzing the applied forces to determine an internal structural feature of the polymer molecule;

wherein the curved outer surfaces each have a radius of curvature of less than one millimeter in at least one direction.

C. A method of sequencing a polymer, comprising:

generating a plurality of droplets in a background fluid, each droplet including a polymer molecule immersed in a foreground fluid and each having an external surface defining an interface between the foreground fluid and the background fluid, wherein the interfaces each have a radius of curvature less than one millimeter in at least one direction;

applying one or more forces to the polymer molecules sufficient to cause the polymer molecules to be translocated across the interfaces;

measuring the applied forces as the polymer molecules are translocated across the interfaces; and

analyzing the applied forces to determine an internal structural feature of the polymer molecules.

D. A method of sequencing a polymer, comprising:

immersing a polymer molecule in a first fluid;

applying a force to the polymer molecule sufficient to cause the polymer molecule to be translocated across a curved interface between the first fluid and a second fluid;

measuring changes in the applied force required to translocate the polymer molecule; and

analyzing the changes to determine an internal structural feature of the polymer molecule;

wherein the curved interface has a radius of curvature of less than one millimeter in at least one direction.

D1. The method of paragraph D, wherein the interface is formed at a tip of a needle, by pushing the first fluid through the needle.

D2. The method of paragraph D, wherein the interface is formed at a tip of a micropipette, by pushing the first fluid through the micropipette.

D3. The method of paragraph D, wherein the interface is formed at an aperture formed in a barrier separating the first fluid from the second fluid.

D4. The method of paragraph D3, wherein the barrier is constructed from graphene.

D5. The method of paragraph D3, wherein the barrier is constructed from a semiconductor material.

D6. The method of paragraph D3, wherein the barrier is constructed from a ceramic material.

D7. The method of paragraph D3, wherein the barrier is constructed from a metal.

D8. The method of paragraph D3, wherein the barrier is constructed from a polymer.

D9. The method of paragraph D8, wherein the polymer is selected from the group consisting of PTFE, polystyrene, polypropylene, and polyethylene.

D10. The method of paragraph D3, wherein the barrier is constructed from a polymer mesh.

D11. The method of paragraph D10, wherein the mesh is constructed from a fluorinated polymer.

D12. The method of paragraph D11, wherein the fluorinated polymer is PTFE.

D13. The method of paragraph D, wherein the radius of curvature of the curved interface changes while the polymer molecule is translocated across the interface.

D14. The method of paragraph D, wherein the radius of curvature of the curved interface is kept constant while the polymer molecule is translocated across the interface.

E. An apparatus for sequencing a polymer, comprising:

a chamber containing an emulsion of droplets suspended in a background fluid, each droplet including a polymer molecule immersed in a foreground fluid;

means for applying a force to the polymer molecules sufficient to cause the polymer molecules to be translocated across an interface between the foreground fluid and the background fluid;

means for measuring the applied forces; and

means for sequencing the polymer molecules based on the measured forces.

F. An apparatus for sequencing a polymer, comprising:

a chamber containing a droplet suspended in a background fluid, the droplet including a polymer molecule immersed in a foreground fluid;

a controllable manipulator configured to be attached to one end of the polymer molecule and to exert a force on the polymer molecule sufficient to pull the polymer molecule through an outer surface of the droplet into the background fluid;

a force sensor configured to measure the force exerted by the manipulator on the polymer molecule; and

a computer processor configured to receive force data from the force sensor and to determine an internal structure of the polymer molecules based on the force data.

F1. The apparatus of paragraph F, wherein the outer surface of the droplet has a radius of curvature less than one millimeter in at least one direction.

F2. The apparatus of paragraph F, wherein the controllable manipulator is a cantilever whose base is attached to a nanopositioning stage.

F3. The apparatus of paragraph F, further comprising means for generating the droplet.

### Surfactants at Interface

G. A method of sequencing a polymer, comprising:

generating a droplet including a polymer molecule immersed in a surrounding liquid, the droplet having a volume defined at least partly by a curved outer surface including a plurality of amphiphilic molecules disposed at the outer surface;

applying a force to the polymer molecule sufficient to cause the polymer molecule to be translocated across the curved outer surface;

measuring the changes in the force required to translocate the molecule; and

analyzing the changes in force to determine an internal structural feature of the polymer molecule.

H. A method of sequencing a polymer, comprising:

generating a plurality of droplets, each including a polymer molecule immersed in a surrounding liquid, each droplet having a volume defined by a curved outer surface including a plurality of amphiphilic molecules disposed at the outer surface;

applying a force to each polymer molecule sufficient to cause the polymer molecule to be translocated across the curved outer surface of the respective droplet;

measuring the changes in force required to translocate the molecule; and

analyzing the changes in force to determine an internal structural feature of the polymer molecule.

I. A method of sequencing a polymer, comprising:

providing a plurality of amphiphilic molecules configured to be disposed at an interface between a first fluid and a second fluid;

immersing a polymer molecule in the first fluid;

applying a force to the polymer molecule sufficient to cause the polymer molecule to be translocated across the interface;

measuring the changes in force required to translocate the molecule; and analyzing the changes in force to determine an internal structural feature of the polymer molecule.

I1. The method of paragraph I, wherein the amphiphilic molecules are members of the Span family.

I2. The method of paragraph I, wherein the amphiphilic molecules are members of the Tween family.

I3. The method of paragraph I, wherein the amphiphilic molecules are Triton X-100 molecules.

I4. The method of paragraph I, wherein the amphiphilic molecules are alkanes with hydrophilic head groups added.

I5. The method of paragraph I, wherein the amphiphilic molecules are cetyl trimethyl ammonium bromide molecules.

I6. The method of paragraph I, wherein the amphiphilic molecules are amphiphilic proteins.

J. An apparatus for sequencing a polymer, comprising:

a chamber containing an emulsion of droplets suspended in a background fluid, each droplet including a polymer molecule immersed in a foreground fluid, and an exterior boundary defining an interface between the foreground fluid and the background fluid, with a plurality of amphiphilic molecules disposed at the interface and configured to interact with the polymer molecule;

means for applying a force to the polymer molecules sufficient to cause the polymer molecules to be translocated across an interface between the foreground fluid and the background fluid;

means for changes in the force required to translocate the molecule; and

means for sequencing the polymer molecules based on the changes in the forces.

K. An apparatus for sequencing a polymer, comprising:

a chamber containing a droplet suspended in a background fluid, the droplet including a polymer molecule immersed in a foreground fluid;

a controllable manipulator configured to be attached to one end of the polymer molecule and to exert a force on the polymer molecule sufficient to pull the polymer molecule through an outer surface of the droplet into the background fluid;

a force sensor configured to measure the force exerted by the manipulator on the polymer molecule; and

a computer processor configured to receive force data from the force sensor and to determine an internal structure of the polymer molecules based on the force data;

wherein a plurality of amphiphilic molecules are disposed at the outer surface of the droplet and configured to interact with the polymer molecule in a predictable manner.

### Solutes in Droplets

L. A method of sequencing a polymer, comprising:

dissolving a plurality of ions in a fluid;

generating a droplet including a polymer molecule immersed in a portion of the fluid, the droplet having a volume defined by a curved outer surface;

applying a force to the polymer molecule sufficient to cause the polymer molecule to be translocated across the curved outer surface;

measuring the changes in force required to translocate the molecule; and

analyzing the changes in force to determine an internal structural feature of the polymer molecule;

wherein an association of the ions with a nucleic acid of the polymer molecule is configured to produce a change in the force required to translocate the molecule.

M. A method of sequencing a polymer, comprising:

generating a plurality of droplets, each droplet including a polymer molecule immersed in a fluid, each droplet having a volume at least partly defined by a curved outer surface, and each droplet including a plurality of ions disposed in the fluid, wherein the ions are configured to exert forces on the polymer molecule;

applying a force to each polymer molecule sufficient to cause the polymer molecule to be translocated across the curved outer surface of the respective droplet;

measuring the changes in force required to translocate the molecule; and analyzing the changes in force to determine an internal structural feature of the polymer molecule.

N. A method of sequencing a polymer, comprising:

generating a plurality of droplets in a background fluid, each droplet including a polymer molecule and a plurality of ions immersed in a foreground fluid, and each having an external surface defining an interface between the foreground fluid and the background fluid;

applying one or more forces to the polymer molecules sufficient to cause the polymer molecules to be translocated across the interfaces;

measuring the changes in forces required to translocate the molecules across the interfaces; and

analyzing the changes in forces to determine an internal structural feature of the polymer molecules;

wherein the ions are configured to interact with the polymer molecules by exerting measurable forces on the polymer molecules as the polymer molecules are translocated across the interfaces.

O. A method of sequencing a polymer, comprising:

immersing a polymer molecule to be sequenced in a first fluid;

applying a force to the polymer molecule to be sequenced sufficient to cause the polymer molecule to be sequenced to be translocated across an interface between the first fluid and a second fluid;

adding a plurality of secondary molecules to at least one of the first fluid and the second fluid, wherein the secondary molecules are configured to interact with the polymer molecule to be sequenced by exerting measurable forces on the polymer molecule to be sequenced as the polymer molecule to be sequenced moves across the interface;

measuring the changes in force to translocate the polymer molecule to be sequenced across the interface; and

analyzing the changes in force to determine an internal structural feature of the polymer molecule to be sequenced.

O1. The method of paragraph O, wherein the secondary molecules are ions.

O2. The method of paragraph O, wherein the secondary molecules are polymers.

O3. The method of paragraph O, wherein the secondary molecules are nucleic acids.

O4. The method of paragraph O, wherein the secondary molecules are peptides or proteins.

O5. The method of paragraph O, wherein the secondary molecules are polysaccharides.

O6. The method of paragraph O, wherein the secondary molecules are added in a concentration sufficient to become entangled with the polymer molecule to be sequenced.

O7. The method of paragraph O, wherein the secondary molecules are added in a concentration insufficient to become entangled with the polymer molecule to be sequenced.

O8. The method of paragraph O, wherein the secondary molecules are cross-linked polymers.

O9. The method of paragraph O, wherein the secondary molecules change from cross-linked polymers to non-cross-linked polymers as the polymer molecule to be sequenced is translocated across the interface.

O10. The method of paragraph O, wherein the secondary molecules change from non-cross-linked polymers to cross-linked polymers as the polymer molecule to be sequenced is translocated across the interface.

O11. The method of paragraph O, wherein the secondary molecules are configured to interact with a specific nucleotide base.

O12. The method of paragraph O, wherein the secondary molecules are configured to interact with a specific nucleotide sequence.

O13. The method of paragraph O, wherein the secondary molecules are dye molecules.

O14. The method of paragraph O, wherein the secondary molecules are aptamers.

P. An apparatus for sequencing a polymer, comprising:

a chamber containing an emulsion of droplets suspended in a background fluid, each droplet including a polymer molecule immersed in a foreground fluid;

means for applying a force to the polymer molecules sufficient to cause the polymer molecules to be translocated across an interface between the foreground fluid and the background fluid;

means for measuring the changes in force required to translocate the molecules; and

means for sequencing the polymer molecules based on the changes in force;

wherein at least one of the background fluid and the foreground fluid contains a plurality of secondary molecules configured to exert measurable forces on the polymer molecules as the polymer molecules are translocated across the respective interfaces.

Q. An apparatus for sequencing a polymer, comprising:

a chamber containing a droplet suspended in a background fluid, the droplet including a polymer molecule immersed in a foreground fluid;

a controllable manipulator configured to be attached to one end of the polymer molecule and to exert a force on the polymer molecule sufficient to pull the polymer molecule through an outer surface of the droplet into the background fluid;

a force sensor configured to measure the force exerted by the manipulator on the polymer molecule; and

a computer processor configured to receive force data from the force sensor and to determine an internal structure of the polymer molecules based on the force data;

wherein the background fluid contains a plurality of secondary molecules configured to exert measurable forces on the polymer molecule as the polymer molecule is pulled through the outer surface of the droplet.

R. An apparatus for sequencing a polymer, comprising:

a chamber containing a droplet suspended in a background fluid, the droplet including a polymer molecule immersed in a foreground fluid;

a controllable manipulator configured to be attached to one end of the polymer molecule and to exert a force on the polymer molecule sufficient to pull the polymer molecule through an outer surface of the droplet into the background fluid;

a force sensor configured to measure the force exerted by the manipulator on the polymer molecule; and

a computer processor configured to receive force data from the force sensor and to determine an internal structure of the polymer molecules based on the force data;

wherein the foreground fluid contains a plurality of secondary molecules configured to exert measurable forces on the polymer molecule as the polymer molecule is pulled through the outer surface of the droplet.

### Oscillating Translocation (Lock-in Measurement)

S. A method of sequencing a polymer, comprising:

generating a plurality of droplets, each droplet including a polymer molecule immersed in a fluid and each droplet having a volume at least partly defined by a curved outer surface;

applying a force to one of the polymer molecules sufficient to cause the polymer molecule to be translocated across the curved outer surface of the respective droplet;

modulating the applied force with at least one predetermined modulation frequency;

measuring the changes in force required to translocate the polymer molecule; and

analyzing the changes in force to determine an internal structural feature of the polymer molecule.

T. A method of sequencing a polymer, comprising:

generating a plurality of droplets in a background fluid, each droplet including a polymer molecule immersed in a foreground fluid, and each droplet having an external surface defining an interface between the foreground fluid and the background fluid;

applying one or more forces to the polymer molecules sufficient to cause the polymer molecules to be translocated across the interfaces;

measuring the changes in force required to translocate the molecules across the interfaces; and

analyzing the changes in force to determine an internal structural feature of the polymer molecules;

wherein the one or more forces include a force modulated at a predetermined modulation frequency.

U. A method of sequencing a polymer, comprising:

immersing a polymer molecule in a first fluid;

applying a translocating force to the polymer molecule sufficient to cause the polymer molecule to be translocated across an interface between the first fluid and a second fluid;

modulating the translocating force with at least one predetermined modulation frequency;

measuring the changes in force required to translocate the molecule across the interface; and

analyzing the changes in force to determine an internal structural feature of the polymer molecule.

U1. The method of paragraph U, wherein modulating the translocating force includes modulating motions of a manipulator which applies the translocating force.

U2. The method of paragraph U, wherein modulating the translocating force includes modulating a position of the interface.

U3. The method of paragraph U2, wherein modulating the translocating force includes modulating the position of the interface at a resonant frequency of a manipulator which applies the translocating force.

U4. The method of paragraph U2, wherein modulating the translocating force includes modulating the position of the interface at a resonant frequency of the interface.

U5. The method of paragraph U4, wherein the resonant frequency of the interface changes as the molecule is translocated across the interface, and wherein modulating the translocating force includes modulating the position of the interface at the changing resonance frequency of the interface.

V. An apparatus for sequencing a polymer, comprising:

a chamber containing an emulsion of droplets suspended in a background fluid, each droplet including a polymer molecule immersed in a foreground fluid;

means for applying a force to the polymer molecules sufficient to cause the polymer molecules to be translocated across an interface between the foreground fluid and the background fluid;

means for modulating the force with at least one predetermined modulation frequency;

means for measuring the changes in force required to translocate the molecule across the interface; and

means for sequencing the polymer molecules based on the measured forces.

W. An apparatus for sequencing a polymer, comprising:

a chamber containing a droplet suspended in a background fluid, the droplet including a polymer molecule immersed in a foreground fluid;

a controllable manipulator configured to be attached to one end of the polymer molecule and to exert a force on the polymer molecule sufficient to pull the polymer molecule through an outer surface of the droplet into the background fluid;

an actuator configured to modulate the force exerted on the polymer molecule at one or more modulation frequencies;

a force sensor configured to measure the force exerted by the manipulator on the polymer molecule; and

a computer processor configured to receive force data from the force sensor and to determine an internal structure of the polymer molecules based on the force data.

X. An apparatus for sequencing a polymer, comprising:

a chamber containing a droplet suspended in a background fluid, the droplet including a polymer molecule immersed in a foreground fluid;

a controllable manipulator configured to be attached to one end of the polymer molecule and to exert a force on the polymer molecule sufficient to pull the polymer molecule through an outer surface of the droplet into the background fluid;

a force sensor configured to measure the force exerted by the manipulator on the polymer molecule; and

a computer processor configured to receive force data from the force sensor and to determine an internal structure of the polymer molecules based on the force data;

wherein the controllable manipulator is further configured to modulate the force exerted on the polymer molecule at one or more modulation frequencies.

### Array-Based Measurements

Y. A method of sequencing a polymer, comprising:

generating a plurality of droplets, each including a polymer molecule immersed in a surrounding liquid, each droplet having a volume defined by a curved outer surface;

simultaneously applying a force to each polymer molecule sufficient to cause the polymer molecule to be translocated across the curved outer surface of the respective droplet;

simultaneously measuring the changes in the forces required to translocate each molecule; and

analyzing the changes in the forces to determine internal structural features of the polymer molecules.

Z. A method of sequencing a polymer, comprising:

generating a plurality of droplets in a background fluid, each droplet including a polymer molecule immersed in a foreground fluid and each having an external surface defining an interface between the foreground fluid and the background fluid;

with an array of controllable manipulators, simultaneously applying forces to the polymer molecules sufficient to cause the polymer molecules to be translocated across the interfaces;

simultaneously measuring the changes in forces required to translocate the molecules across the interfaces; and

analyzing the changes in forces to determine an internal structural feature of the polymer molecules.

AA. An apparatus for sequencing a polymer, comprising:

a chamber containing an emulsion of droplets suspended in a background fluid, each droplet including a polymer molecule immersed in a foreground fluid;

means for simultaneously applying forces to the polymer molecules sufficient to cause the polymer molecules to be translocated across an interface between the foreground fluid and the background fluid;

means for simultaneously measuring the applied forces; and

means for sequencing the polymer molecules based on the measured forces.

BB. An apparatus for sequencing a polymer, comprising:

a chamber containing a plurality of polymer molecules, each immersed in a foreground fluid having an outer surface;

an array of controllable manipulators, each configured to be attached to an end of one of the polymer molecules and to exert a force on the polymer molecule sufficient to pull the polymer molecule through the outer surface of the foreground fluid;

an array of force sensors, each configured to measure the force exerted by one of the manipulators on the associated polymer molecule; and

a computer processor configured to receive force data from the force sensors and to determine an internal structure of the polymer molecules based on the force data.

BB1. The apparatus of paragraph BB, wherein each polymer molecule is contained in a droplet of the foreground fluid immersed in a background fluid.

BB2. The apparatus of paragraph BB, wherein the chamber includes a plurality of sample wells, and each polymer molecule is contained in one of the sample wells along with an associated amount of the foreground fluid.

Pulling from Denaturing Conditions

CC. A method of sequencing a polymer chain, comprising:

immersing a biological macromolecule in a first fluid;

denaturing the biological macromolecule into single polymer chains;

applying a force to one of the polymer chains sufficient to cause the polymer chain to be translocated across a curved interface between the first fluid and a second fluid;

measuring the applied force as the polymer chain is translocated; and

analyzing the applied force to determine a molecular sequence of the polymer chain.

CC1. The method of paragraph CC, wherein denaturing the biological macromolecule includes applying heat.

CC1a. The method of paragraph CC1, further comprising adding a solute to the first fluid to increase a boiling point of the first fluid.

CC1b. The method of paragraph CC1, further comprising applying pressure to the first fluid to increase a boiling point of the first fluid.

CC2. The method of paragraph CC, wherein denaturing the biological macromolecule includes exposing the biological macromolecule to sodium hydroxide.

CC3. The method of paragraph CC, wherein denaturing the biological macromolecule includes exposing the biological macromolecule to urea.

CC4. The method of paragraph CC, wherein at least one of the first fluid and the second fluid is a liquid having a pH sufficient to denature the biological macromolecule.

CC5. The method of paragraph CC, wherein denaturing the biological macromolecule includes exposing the biological macromolecule to an organic solvent.

CC6. The method of paragraph CC, wherein denaturing the biological macromolecule includes exposing the biological macromolecule to potassium hydroxide.

DD. An apparatus for sequencing a polymer, comprising:

a chamber containing an emulsion of droplets suspended in a background fluid, each droplet including a biological macromolecule immersed in a foreground fluid;

means for denaturing the biological macromolecules into single polymer chains;

means for applying forces to the polymer chains sufficient to cause the polymer molecules to be translocated across an interface between the foreground fluid and the background fluid;

means for measuring the applied forces; and

means for sequencing the polymer chains based on the measured forces.

EE. An apparatus for sequencing a polymer chain, comprising:

a chamber containing a droplet suspended in a background fluid, the droplet including a denatured biological macromolecule immersed in a foreground fluid, wherein the denatured biological macromolecule includes a polymer chain;

a controllable manipulator configured to be attached to one end of the polymer chain and to exert a force on the polymer chain sufficient to pull the polymer chain through an outer surface of the droplet into the background fluid;

a force sensor configured to measure the force exerted by the manipulator on the polymer chain; and

a computer processor configured to receive force data from the force sensor and to determine an internal structure of the polymer molecules based on the force data.

FF. An apparatus for sequencing a polymer chain, comprising:

a chamber containing a plurality of biological macromolecules suspended in a first fluid and exposed to a denaturing agent, and a second fluid separated from the first fluid by an interface;

a controllable manipulator configured to be attached to one end of a polymer chain resulting from exposure of the biological macromolecules to the denaturing agent and to exert a force on the polymer chain sufficient to pull the polymer chain at least partially through the interface and into the second fluid;

a force sensor configured to measure the force exerted by the manipulator on the polymer chain; and

a computer processor configured to receive force data from the force sensor and to determine a molecular sequencing of the polymer chain based on the force data.

Sequencing with Magnetic Straightening Force

GG. A method of sequencing a polymer, comprising:

immersing a polymer molecule having first and second ends in a first fluid;

attaching the first end of the polymer molecule to a magnetic trap;

applying a force to the second end of the polymer molecule sufficient to cause a portion of the polymer molecule to be translocated across an interface between the first fluid and a second fluid;

measuring the applied force as the polymer molecule is translocated; and

analyzing the applied force to determine an internal structural feature of the polymer molecule.

GG1. The method of paragraph GG, wherein the magnetic trap is a magnetic bead, and further comprising applying a magnetic field to the magnetic bead which tends to hold the magnetic bead within the first fluid.

GG2. The method of paragraph GG, wherein the magnetic trap is a nonspecific attachment of the first end of the polymer molecule to a magnetic element.

GG3. The method of paragraph GG, wherein the magnetic trap is configured to move the polymer laterally in the interface.

HH. An apparatus for sequencing a polymer, comprising:

a chamber containing a plurality of polymer molecules and a plurality of magnetic beads suspended in a first fluid, each polymer molecule having a first end attached to one of the magnetic beads;

means for applying forces to the magnetic beads tending to retain the magnetic beads within the first fluid;

means for applying forces to a second end of each polymer molecule sufficient to cause a portion of each polymer molecule to be translocated across an interface between the first fluid and a second fluid;

means for measuring the forces applied to the second ends of the polymer molecules; and

means for sequencing the translocated portions of the polymer molecules based on the measured forces.

II. An apparatus for sequencing a polymer, comprising:

a chamber containing a polymer molecule and a magnetic bead suspended in a first fluid, and further containing a second fluid separated from the first fluid by a fluid interface, wherein one end of the polymer molecule is attached to the magnetic bead;

a magnetic field generator configured to generate a magnetic field tending to retain the magnetic bead with the first fluid;

a controllable manipulator configured to be attached to another end of the polymer molecule and to exert a force on the polymer molecule sufficient to pull a portion of the polymer molecule through the interface and into the second fluid;

a force sensor configured to measure the force exerted by the manipulator on the polymer molecule; and

a computer processor configured to receive force data from the force sensor and to determine an internal structure of the polymer molecules based on the force data.

Pulling into Air

JJ. A method of sequencing a polymer, comprising:

generating a droplet including a polymer molecule immersed in a surrounding liquid, the droplet having a volume defined by a curved outer surface and suspended in air;

applying a force to the polymer molecule sufficient to cause the polymer molecule to be translocated across the curved outer surface into the air;

measuring the applied force as the polymer molecule is translocated; and

analyzing the applied force to determine an internal structural feature of the polymer molecule.

JJ1. The method of paragraph JJ, wherein the air has a relative humidity less than 50%.

JJ2. The method of paragraph JJ, wherein the air has a relative humidity less than 20%.

JJ3. The method of paragraph JJ, wherein the air has a relative humidity less than 10%.

JJ4. The method of paragraph JJ, wherein the air has a relative humidity less than 5%.

JJ5. The method of paragraph JJ, wherein the air has a relative humidity less than 1%.

KK. A method of sequencing a polymer, comprising:

generating a plurality of droplets in a background of air, each including a polymer molecule immersed in a surrounding liquid, each droplet having a volume defined by a curved outer surface;

applying a force to each polymer molecule sufficient to cause the polymer molecule to be translocated across the curved outer surface of the respective droplet and into the air;

measuring the applied force as each polymer molecule is translocated; and

analyzing the applied forces to determine an internal structural feature of the polymer molecule.

LL. A method of sequencing a polymer, comprising:

generating a plurality of droplets in a background of air, each droplet including a polymer molecule immersed in a foreground fluid and each having an external surface defining an interface between the foreground fluid and the air;

applying one or more forces to the polymer molecules sufficient to cause the polymer molecules to be translocated across the interfaces and into the air;

measuring the applied forces as the polymer molecules are translocated across the interfaces; and

analyzing the applied forces to determine an internal structural feature of the polymer molecules.

MM. A method of sequencing a polymer, comprising:

immersing a polymer molecule in a first fluid;

applying a force to the polymer molecule sufficient to cause the polymer molecule to be translocated across a curved interface between the first fluid and a background of air having a known relative humidity;

measuring the applied force as the polymer molecule is translocated; and

analyzing the applied force to determine an internal structural feature of the polymer molecule.

NN. An apparatus for sequencing a polymer, comprising:

a chamber containing an emulsion of droplets suspended in air, each droplet including a polymer molecule immersed in a foreground fluid;

means for applying a force to each of the polymer molecules sufficient to cause the polymer molecules to be translocated across an interface between the foreground fluid and into the air;

means for measuring the applied forces; and

means for sequencing the polymer molecules based on the measured forces.

OO. An apparatus for sequencing a polymer, comprising:

a chamber containing a droplet suspended in air, the droplet including a polymer molecule immersed in a foreground fluid;

a controllable manipulator configured to be attached to one end of the polymer molecule and to exert a force on the polymer molecule sufficient to pull the polymer molecule through an outer surface of the droplet into the air;

a force sensor configured to measure the force exerted by the manipulator on the polymer molecule; and

a computer processor configured to receive force data from the force sensor and to determine an internal structure of the polymer molecules based on the force data.

What is claimed is:

1. A method of sequencing a polymer, comprising:
immersing a polymer molecule in a first fluid;
translocating the polymer molecule across an interface between the first fluid and a second fluid;
keeping a radius of curvature of the interface constant while the polymer molecule is translocated across the interface;
measuring changes in a force associated with the translocation of the polymer molecule while translocating the polymer molecule; and
analyzing the changes to determine an internal structural feature of the polymer molecule.

2. The method of claim 1, wherein the interface has a radius of curvature of less than one millimeter in at least one direction.

3. The method of claim 2, wherein the radius of curvature of the interface is less than one micrometer in at least one direction.

4. The method of claim 1, wherein the first fluid is a liquid and the second fluid is a vapor.

5. The method of claim 1, wherein the interface is formed at a tip of a needle by pushing the first fluid through the needle.

6. The method of claim 1, wherein the interface is formed at an aperture formed in a barrier separating the first fluid from the second fluid.

7. The method of claim 1, wherein the barrier comprises a polymeric material.

8. The method of claim 1, wherein the second fluid comprises air.

9. A method of sequencing a polymer, comprising:
immersing a polymer molecule in a first fluid;
translocating a polymer molecule across an interface between the first fluid and a second fluid;
modulating the translocation with at least one predetermined modulation frequency;
measuring the changes in force required to translocate the molecule across the interface; and
analyzing the changes in force to determine an internal structural feature of the polymer molecule.

10. The method of claim 9, wherein modulating the translocation includes modulating a position of the interface.

11. The method of claim 9, wherein modulating the translocation includes modulating motions of a manipulator which causes the translocation.

12. The method of claim 11, wherein the manipulator comprises a cantilever attached to the polymer molecule and driven by a piezoelectric motor.

13. The method of claim 12, wherein modulating the translocation includes modulating the position of the interface at a resonant frequency of a manipulator which causes the translocation.

14. The method of claim 12, wherein modulating the translocation includes modulating the position of the interface at a resonant frequency of the interface.

15. The method of claim 14, wherein the resonant frequency of the interface changes as the molecule is translocated across the interface, and wherein modulating the translocation includes modulating the position of the interface at the changing resonance frequency of the interface.

16. A method of sequencing a polymer, comprising:
attaching a polymer to a manipulator;
detecting attachment of the polymer to the manipulator;
immersing the polymer in a first fluid;
applying a force to the polymer molecule with the manipulator sufficient to cause the polymer molecule to be translocated across an interface between the first fluid and a second fluid;
measuring changes in the force applied by the manipulator required to translocate different portions of the polymer molecule; and
analyzing the changes in the required force to determine an internal structural feature of the polymer molecule.

17. The method of claim 16, wherein detecting attachment of the polymer to the manipulator includes measuring a force applied with the manipulator when the manipulator is a desired height above the interface, and determining that the force is within a predetermined range.

18. The method of claim 16, wherein applying the force to the polymer molecule includes repeatedly translocating the molecule across the interface in a flossing motion.

19. The method of claim 18, wherein measure changes in the force applied includes averaging repeated for profile measurements corresponding to repeatedly translocating the molecule.

20. The method of claim 18, wherein repeatedly translocating the molecule is performed for the same known portion of the molecule.
